(19) United States
(12) Patent Application Publication
(10) Pub. No.: US 2004/0087772 A1
(43) Pub. Date: May 6, 2004

(54) Xage-1, a gene expressed in multiple cancers, and uses thereof

(76) Inventors: Ira Pastan, Potomac, MD (US); Xiu Fen Liu, Clarksville, MD (US); Tapan K Bera, Gaithersburg, MD (US); Byungkook Lee, Potomac, MD (US); Kristi A England, Silverspring, MD (US)

Correspondence Address: Laurence J Hyman, Townsend and Townsend and Crew, 8th Floor, Two Embarcadero Center, San Francisco, CA, 94111-3834, US

(21) Appl. No.: 10/363,233

(22) PCT Filed: Aug. 31, 2001
(86) PCT No.: PCT/US01/27258

(51) Int. Cl. A61K 38/17; C07K 14/74; C07H 21/04; A61K 39/395; C07K 16/28

(52) U.S. Cl. 530/350; 530/388.22; 514/12; 424/144.1

(57) ABSTRACT

The invention relates to the surprising discovery that XAGE-1 is translated as two proteins, a 9 kD protein, termed p9, and a 16.3 kD protein, termed p16. The invention further relates to the surprising discovery that XAGE-1 is expressed in a number of important human cancers, specifically: prostate cancer, lung cancer, ovarian cancer, breast cancer, glioblastoma, pancreatic cancer, T cell lymphoma, melanoma, and histocytic lymphoma. The proteins p9 and p16, immunogenic fragments thereof, analogs of these proteins, and nucleic acids encoding these proteins, fragments, or analogs, can be administered to persons with XAGE-1 expressing cancers to raise or augment an immune response to the cancer. The invention further provides nucleic add sequences encoding the proteins, as well as expression vectors, host cells, and antibodies to the proteins. Further, the invention provides immunoconjugates that comprise an antibody to p16 or to p9, and an effector molecule, such as a label, a radioisotope, or a toxin. The invention also provides methods of inhibiting the growth of XAGE-1 expressing cells by contacting them with immunoconjugates comprising an anti-p9 or p16 antibody and a toxic moiety. Further, the invention provides kits for detecting the presence of p9 or p16 in a sample.

### CROSS-REFERENCES TO RELATED APPLICATIONS

This application claims priority from U.S. Provisional Patent Application No. 60/229,684, filed Sep. 1, 2000, the contents of which are incorporated for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### FIELD OF THE INVENTION

This invention relates to the discovery that the gene known as XAGE-1 is expressed in a number of cancers, that the gene and its expressed proteins can be used to detect the presence of XAGE-1-expressing cancers, and that the proteins encoded by the gene can be used to augment immune responses to such a cancer. Additionally, the invention relates to the use of immunoconjugates bearing toxic moieties for the manufacture of medicaments to inhibit the growth of XAGE-1-expressing cancers, and the use of such immunoconjugates to inhibit the growth of XAGE-1-expressing cancer cells.

### BACKGROUND OF THE INVENTION

Large numbers of expressed sequence tags (ESTs) have been cloned from various tissues and cancers (Adams, M. D. et al., Nature, 377:3-174 (1995); Adams, M. D. et al., Science, 252:1651-1656 (1991)). Each cDNA clone or EST sequence is generated from a single transcript. The frequency and distribution of the many different transcripts in a given tissue depends oh the level of gene expression. Therefore, a particular gene expression pattern can be frequently predicted by analysis of the frequency and specificity of various EST sequences. A computer screening strategy has been reported that identified genes that are preferentially expressed in prostate or prostate tumors (Liu, X. et al., Biochem. Biophys. Res. Comm., 264:833-839 (1999); Vasmatzis, G. et al., Proc. Natl. Acad. Sci. USA., 95:300-304 (1998); Essand, M. et al., Proc. Natl. Acad. Sci. USA., 96:9287-9292 (1999)). From this screen, several genes were identified including a novel gene, PAGE4 (previously named PAGE1) (Brinkmann, U. et al., Proc. Natl. Acad. Sci. USA., 95:10757-10762 (1998)), and a set of XAGEs genes (Brinkmann, U. et al., Cancer Res., 59:1445-1448 (1999) (hereafter referred to as “Brinkmann 1999”)), which are related to the GAGE, MAGE family of melanoma associated cancer-testis antigens.

Cancer-testis (CT) antigens are a distinct class of differentiation antigens that have a restricted pattern of expression in normal tissues(De Smet, C. et al., Eye., 11:243-248(1997); Chen, Y. T. Cancer J. Sci. Am., 5:16-17 (1999); Gillespie, A. et al., Br. J. Cancer., 78:816-821 (1998)). Some thoroughly studied CT antigens are MAGE, BAGE, GAGE and LAGE/NY-ESO-1(Chen, Y. T. Cancer J. Sci. Am., 5:16-17 (1999); Gillespie, A. et al., Br. J. Cancer., 78:816-821 (1998); Lucas, S. et al., Cancer Res., 58:743-752 (1998); Jungbluth, A. A. et al., Int. J. Cancer., 85:460-465 (2001); Chen, Y. T. et al., Proc. Natl. Acad. Sci. USA., 95:6919-6923 (1998); Boel, P. et al., Immunity., 2:167-175 (1995); Backer, O. et al., Cancer Res., 59:3157-3165 (1991); De Plaen, E. et al., Immunogenetics. 40:360-369 (1994); Chen, Y. T. et al., Cell Genet., 79:237-240 (1997)). These genes are primarily expressed in the primitive germ cells, spermatogonia, and in the normal testis. Malignant transformation is often associated with activation or derepression of silent CT genes, and this results in the expression of CT antigens in a variable proportion of a wide range of human tumors. Recently, several additional members were added to the CT antigen family. These include various PAGEs, PRAME, SSX, SCP-1, CT7 and MAGEC1 and MAGED1 (Brinkmann, U. et al., Proc. Natl. Acad. Sci. USA., 95:10757-10762 (1998); Lucas, S. et al., Cancer Res., 58:743-752 (1998); Gure, A. O. et al., Int. J. Cancer., 85:726-732 (2000); Tureci, O. et al., Int. J. Cancer., 77:19-23 (1998); Tureci, O. et al., Proc. Natl. Acad. Sci. USA., 95:5211-5216 (1998); Pold, M. et al., Genomics., 59:161-167 (1999); Watari, K. et al., FEBS Lett., 466: 367-371 (2000)). Identification of new CT antigens or new family members continues to be pursued in the cancer research field.

Three related genes, termed XAGEs, were recently identified by homology walking using the dbEST database (Briann 1999). ESTs of the XAGE group were found in various cDNA libraries. The XAGE-1 cluster contained ESTs from testis, germ cell tumors, and from some relatively rare tumors of bone and muscle most frequently found in children: Ewing's sarcoma, and alveolar rhabdomyosarcoma The authors of Brinkmann 1999 reported, however, that there appeared to be two reading frames, and that the second did not contain a start codon until about halfway through the sequence. Due to the uncertainty with translation, the authors were unable to report a protein encoded by the gene they named “XAGE-1.” Accordingly, Brinkmann 1999 did not report a sequence for XAGE-1 or of any proteins it might encode.

### BRIEF SUMMARY OF THE INVENTION

This invention relates to the discovery of two proteins expressed from the XAGE-1 gene, to uses of the proteins and of the nucleic acid encoding them, to antibodies against the proteins, as well as to the use of the proteins or to nucleic acids encoding them for the manufacture of medicaments to XAGE-1 expressing cancers.

Specifically, the invention provides an isolated polypeptide comprising an amino acid sequence selected from the group consisting of a xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9. In some embodiments, the isolated polypeptide comprises the sequence of p9. The invention further provides compositions comprising an isolated polypeptide selected from the group consisting of a xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9, and a pharmaceutically acceptable carrier.

In another group of embodiments, the invention provides an isolated, recombinant nucleic acid molecule comprising a nucleotide sequence encoding a polypeptide having an amino acid sequence selected from the group of the amino acid sequence of an xage-1 p9 protein (“p9,” SEQ ID NO:2), an immunogenic fragment thereof a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9. In a preferred embodiment, the isolated, recombinant nucleic acid molecule encodes a polypeptide comprising the sequence of xage-1 p9.

The invention further provides expression vectors comprising a promoter operatively linked to a nucleotide sequence encoding a polypeptide selected from the group consisting of: xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9. Additionally, the invention provides host cells expressing any of these expression vectors.

In an important group of embodiments, the invention provides the use of an isolated polypeptide comprising an amino acid sequence selected from the group consisting of a xage-1 p9 protein (“p9” (SEQ ID NO:2)), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9 for the manufacture of a medicament for activating T lymphocytes against cells expressing xage-1 p9. In a preferred embodiments, the use is of an isolated polypeptide comprising the sequence of xage-1 p9. In other preferred embodiments, the use is for the manufacture of a medicament for activating T lymphocytes against cells expressing xage-1 p9 other than cells of Ewing's sarcoma and alveolar rhabdomyosarcoma In particularly preferred embodiments, the cells expressing XAGE-1 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells. Among lung cancer cells, lung cancer cells selected from the group of small cell carcinoma cells, non-small cell carcinoma cells, squamous cell carcinoma cells, and adenocarcinoma cells are particularly preferred. In especially preferred embodiments, the isolated, recombinant nucleic acid molecule encodes the sequence of xage-1 p9 (SEQ ID NO:2).

The invention further provides the use of an isolated, recombinant nucleic acid molecule comprising a nucleotide sequence encoding a polypeptide having the amino acid sequence of an xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9, for the manufacture of a medicament for activating T lymphocytes against cells expressing xage-1 p9 other than cells of Ewing's sarcoma and alveolar rhabdomyosarcoma. In preferred embodiments, the cells expressing xage-1 p9 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells. In particularly preferred embodiments, the isolated, recombinant nucleic acid molecule encodes xage-1 p9 (SEQ ID NO:2).

In another group of embodiments, the invention provides a method of activating T lymphocytes against cells expressing xage-1 p9 (SEQ ID NO:2), the method comprising administering to a subject a composition, which composition is selected from the group consisting of: an isolated polypeptide having the amino acid sequence of xage-1 p9, an immunogenic fragment thereof, a polypeptide with at least 90% sequence identity to xage-1 p9 and which is specifically recognized by an antibody which specifically recognizes xage-1 p9, a polypeptide which has at least 90% sequence identity with xage-1 p9 and which, when processed and presented in the context of Major Histocompatibility Complex molecules, activates T lymphocytes against cells which express xage-1 p9, an isolated nucleic acid encoding one of these polypeptides, an antigen presenting cell pulsed with a polypeptide comprising an epitope of xage-1 p9, an antigen presenting cell sensitized in vitro to xage-1 p9, an antigen presenting cell sensitized in vitro to an immunogenic fragment of xage-1 p9, an antigen presenting cell sensitized in vitro to a polypeptide with at least 90% sequence identity to xage-1 p9 which is specifically recognized by an antibody which specifically recognizes xage-1 p9, and an antigen presenting cell sensitized in vitro to polypeptide which has at least 90% sequence identity with xage-1 p9 which, when processed and presented in the context of Major Histocompatibility Complex molecules, activates T lymphocytes against cells which express xage-1 p9. In preferred embodiments, the method comprises administering to the subject xage-1 p9 or an immunogenic fragment thereof. In particularly preferred embodiments, the composition is administered to a subject who suffers from a cancer selected from prostate cancer cells, lung cancer cells, ovarian cancer cells, breast cancer cells, glioblastoma cells, pancreatic cancer cells, T cell lymphoma cells, melanoma cells, and histocytic lymphoma cells. With respect to lung cancer cells, a lung cancer selected from the group consisting of small cell carcinoma, non-small cell carcinoma, squamous cell carcinoma, and adenocarcinoma is preferred. In some embodiments, the composition is administered to a subject suffering from a cancer selected from the group consisting of Ewing's sarcoma, rhabdomyosarcoma and osteosarcoma

In some embodiments, the method comprises sensitizing CD8+ cells in vitro to an epitope of an xage-1 p9 protein (SEQ ID NO:2) and administering the sensitized cells to the subject. Further, the method may comprises co-administering to the subject an immune adjuvant selected from non-specific immune adjuvants, subcellular microbial products and fractions, haptens, immunogenic proteins, immunomodulators, interferons, thymic hormones and colony stimulating factors. The method may also comprises administering an antigen presenting cell pulsed with a polypeptide comprising an epitope of xage-1 p9 (SEQ ID NO:2). In some embodiments, the method may comprise administering a nucleic acid sequence encoding polypeptide comprising an epitope of xage-1 p9 (SEQ ID NO:2), which nucleic acid is in a recombinant virus. In some embodiments, the method may comprise administering a nucleic acid sequence encoding a polypeptide comprising an epitope of an xage-1 p9 protein (SEQ ID NO:2). The method may comprise immunizing the subject with a expression vector that expresses a polypeptide comprising an epitope of an xage-1 p9 protein (SEQ ID NO:2), which expression vector is in an autologous recombinant cell. The CD8+ cells used in the above methods can be T_{C} cells. The T_{C} cells can be tumor infiltrating lymphocytes.

In another group of embodiments, the invention provides methods for determining whether a subject has an xage-1 p9 expressing cancer, comprising taking a cell sample from said subject from a site other than the testes, and determining whether a cell in said sample contains a nucleic acid transcript encoding xage-1 p9 (SEQ ID NO:2), or detecting xage-1 p9 produced by translation of the transcript, whereby detection of the transcript or of the protein in said sample indicates that the subject has an xage-1 p9 expressing cancer. Methods involving detection of the transcript can comprise contacting RNA from the cell with a nucleic acid probe that specifically hybridizes to the transcript under hybridization conditions, and detecting hybridization. The methods involving detection of the protein may also comprise disrupting the cell and contacting a portion of the cell contents with a chimeric molecule comprising a targeting moiety and a detectable label, wherein the targeting moiety specifically binds to xage-1 p9 (SEQ ID NO:2), and detecting the label bound to the xage-1 p9. In some embodiments, the cell is taken from a lymph node.

In a major group of embodiments, the invention provides an isolated polypeptide comprising an amino acid sequence selected from the group consisting of a xage-1 p16 protein (“p16,” SEQ ID NO:4), an immunogenic fragment thereof a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16. In preferred embodiments, the polypeptide comprises the sequence of p16. The invention further provides compositions of any of these polypeptides and a pharmaceutically acceptable carrier.

In a further set of embodiments, the invention provides isolated, recombinant nucleic acid molecules comprising a nucleotide sequence encoding a polypeptide selected from the group of one having the amino acid sequence of an xage-1 p16 protein (“p16”, (SEQ ID NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16. In preferred embodiments, the isolated, recombinant nucleic acid molecule encodes a polypeptide having the sequence of xage-1 p16.

The invention further provides expression vectors comprising an isolated, recombinant nucleic acid molecule comprising a nucleotide sequence encoding a polypeptide selected from the group of one having the amino acid sequence of an xage-1 p16 protein (“p16”, (SEQ ID NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16. In preferred embodiments, the isolated, recombinant nucleic acid molecule encodes a polypeptide having the sequence of xage-1 p16, operatively linked to a promoter.

In another group of embodiments, the invention provides the use of an isolated polypeptide comprising an amino acid sequence selected from the group consisting of the amino acid sequence of a xage-1 p16 protein (“p16” (SEQ ID NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16, for the manufacture of a medicament for activating T lymphocytes against cells expressing xage-1 p16. In preferred embodiments, the cells expressing xage-1 p16 are cancer cells. In more preferred forms, the cancer cells are of cancers other than Ewing's sarcoma or alveolar rhabdomyosarcoma In even more preferred forms, the cells expressing xage-1 p16 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells. With regard to lung cancer cells, small cell carcinoma cells, non-small cell carcinoma cells, squamous cell carcinoma cells, and adenocarcinoma cells are particularly preferred. In especially preferred embodiments, the nucleic acid molecule encodes a polypeptide comprising the sequence of xage-1 p16 (SEQ ID NO:4).

The invention further relates to the use of an isolated, recombinant nucleic acid molecule comprising a nucleotide sequence encoding a polypeptide having the amino acid sequence of an xage-1 p16 protein (“p16” (SEQ ID NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16, for the manufacture of a medicament for activating T lymphocytes against cells expressing xage-1 p16. In preferred embodiments, the cells expressing xage-1 p16 are cancer cells. In more preferred forms, the cancer cells are of cancers other than Ewing's sarcoma or alveolar rhabdomyosarcoma. In even more preferred forms, the cells expressing xage-1 p16 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells. With regard to lung cancer cells, small cell carcinoma cells, non-small cell carcinoma cells, squamous cell carcinoma cells, and adenocarcinoma cells are particularly preferred. In especially preferred embodiments, the nucleic acid molecule encodes a polypeptide comprising the sequence of xage-1 p16 (SEQ ID NO:4).

In another group of embodiments, the invention provides antibodies that specifically binds to an epitope of a protein selected from the group consisting of xage-1 p16 protein (SEQ ID NO:4), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16. In preferred embodiments, the protein is xage-1 p16 (SEQ ID NO:4). The antibody may be fused or conjugated to a therapeutic moiety or a detectable label. In preferred embodiments, the therapeutic moiety is a toxic moiety. The toxic moiety may be selected from the group consisting of ricin A, abrin, ribotoxin, ribonuclease, saporin, calicheamycin, diphtheria toxin or a subunit thereof, Pseudomonas exotoxin, a cytotoxic portion thereof, a mutated Pseudomonas exotoxin, a cytotoxic portion thereof, and botulinum toxins A through F, pokeweed antiviral toxin or a cytotoxic fragment thereof, and bryodin 1 or a cytotoxic fragment thereof. In preferred embodiments, the toxic moiety is a Pseudomonas exotoxin or a cytotoxic fragment thereof. In particularly preferred embodiments, the Pseudomonas exotoxin is selected from the group consisting of PE35, PE38, PE4E, and PE40. The detectable label may be a radiolabel.

In yet another group of embodiments, the invention provides methods of inhibiting the growth of a cancer cell expressing xage-1 p16 (SEQ ID NO:4) on an exterior surface, comprising contacting the cell with an immunoconjugate comprising a therapeutic moiety and a targeting moiety, the targeting moiety comprising a polypeptide comprising an antibody which specifically binds to an epitope of xage-1 p16, wherein said binding permits the therapeutic moiety to inhibit the growth of the cell. The therapeutic moiety can be a drug. In some embodiments, the therapeutic moiety is a radioisotope. In preferred embodiments, the therapeutic moiety is a toxin. The toxin can be selected from the group consisting of ricin A, abrin, ribotoxin, ribonuclease, saporin, calicheamycin, diphtheria toxin or a subunit thereof, Pseudomonas exotoxin, a cytotoxic portion thereof, a mutated Pseudomonas exotoxin, a cytotoxic portion thereof, and botulinum toxins A through F, pokeweed antiviral toxin or a cytotoxic fragment thereof, and bryodin 1 or a cytotoxic fragment thereof. In preferred embodiments, the toxin is a modified or mutated Pseudomonas exotoxin or cytotoxic fragment thereof.

The invention further provides kits for the detection of an xage-1 p16-expressing cancer in a sample, said kit comprising a container and an antibody which specifically recognizes xage-1 p16 (SEQ ID NO:4). In preferred embodiments, the cancer is one other than Ewing's sarcoma or alveolar rhabdomyosarcoma

### BRIEF DESCRIPTION OF THE DRAWINGS

FIG. 1. FIG. 1A. Diagram of the XAGE-1 transcripts. The complete XAGE-1 sequence shown, with untranslated 5′ and 3′ ends, is SEQ ID NO:5. The polyadenylation signal is italicized and in bold. The translation stop and start codons are indicated in bold. Primers are indicated by arrows and by name, and the transcriptional start sites are indicated by “star burst” symbols above the nucleotide sequence. Intron/exon boundaries are indicated by vertical lines capped with a horizontal line (i.e., a “T” shaped symbol).

FIGS. 1B and 1C: Primer extension analysis of XAGE-1. FIG. 1B. Primer extension analysis was performed using the primer Xagext.4 to define the 5′ most transcriptional start site. FIG. 1C. Primer extension analysis was performed using the primer Xagext.3 to define the position of the downstream start site. The primer extension products are indicated by the arrows. The sequencing ladder is shown on the right. For both FIG. 1B and FIG. 1C, the lanes were as follows: 1, no RNA; 2, testis RNA; 3, Ewing's sarcoma cell line TC71 RNA.

FIG. 2. In situ hybridization analysis of XAGE-1 expression in normal breast cells, normal prostate cells, breast cancer cells, and prostate cancer cells. The top row shows the results of probes of breast cells. The left photo in the top row shows breast tissue probed with pBlueScript containing no insert, as a negative control. The middle photo in the top row shows a section of normal breast tissue probed using pBlueScript containing XAGE-1. The right hand photo in the top row shows a section of a breast cancer probed using pBlueScript containing XAGE-1. The bottom row shows the results of probes of prostate cells. The left photo in the bottom row shows prostate tissue probed with pBlueScript containing no insert, as a negative control. The middle photo in the bottom row shows a section of normal prostate tissue probed using pBlueScript containing XAGE-1. The right hand photo in the bottom row shows a section of prostate cancer probed using pBlueScript containing XAGE-1.

### DETAILED DESCRIPTION

### Introduction

Surprisingly, the gene termed XAGE-1 encodes two proteins, an intracellular protein with a weight of approximately 9 kD (“xage-1 p9” or “p9”), and a membrane-associated 146-amino acid protein with a putative molecular weight of approximately 16.3 kD (‘xage-1 p16” or “p16”). The two proteins are encoded by the same reading frame of the RNA, but start at alternative start codons. The start codon of the 9 kD protein is all the more surprising since it is initiated from an ATG 103 bp downstream of the first translational start codon. Nonetheless, the 9 kD form is preferentially expressed by cells of the 293T (human embryonic kidney) line in transfection assays using a plasmid containing XAGE-1 cDNA.

It has now also surprisingly been discovered that XAGE-1, which had been found only in EST libraries of normal testes, of certain relatively rare muscle and bone cancers, and of germ line tumors, is expressed in a number of cancers which are much more common, and which account for a substantial portion of human mortality from cancer. XAGE-1 expression has now been detected, for example, in breast lobular carcinomas and breast infiltrating ductal carcinomas. XAGE-1 is abundantly expressed in numerous lung carcinomas, including squamous cell carcinomas, adenocarcinomas, and bronchiolo-alveolar adenocarcinoma. In addition, XAGE-1 has been found to be expressed kidney transitional cell carcinoma, rectum adenosquamous carcinoma, chronic myelogenous leukemia cell line K562 and lung carcinoma cell line A549.

Normal breast samples were found to express XAGE-1 either weakly, or not at all. In contrast, two thirds of breast cancer cDNA samples showed more abundant PCR products than PCR products from the normal samples, showing that XAGE-1 is up-regulated in breast cancer. It was also expressed in all the small cell and non-small cell tumors of the lung tested, as well as in two thirds of the squamous cell carcinomas and adenocarcinomas studied. Moreover, XAGE-1 expression was found in two thirds of the prostate cancer cell lines studied. XAGE-1 expression was also noted in a pancreatic adenocarcinoma. In contrast, among normal tissues, the gene is expressed at high levels only in the testes.

XAGE-1 has therefore now been found to be widely expressed in common cancers accounting for a substantial portion of overall cancer mortality, as opposed to the original findings that it was expressed only in relatively uncommon cancers primarily found in children. The current findings elevate XAGE-1 from relatively modest interest to a focus of attention as a therapeutic and diagnostic target.

The presence of xage-1 p16 and especially of xage-1 p9 protein in many prostate cancer cells, and cells of breast cancers, of lung cancers (including squamous cell carcinoma, small cell carcinoma, non-small cell carcinoma, and adenocarcinoma), in cells of T cell and histiocytic lymphomas, in melanoma cells, in glioblastoma cells, and in cells of ovarian cancer creates a number of opportunities for in vitro and in vivo uses. First, antibodies raised against the proteins can be used in in vitro assays to detect the presence of cells expressing XAGE-1 in a sample. For example, detection of significant levels of XAGE-1 or of xage-1 p9 or p16 in cells taken in a lung biopsy would be indicative of the presence of a XAGE-1-expressing cancer in the subject since XAGE-1 is expressed only at very low levels in normal lung tissue. Conveniently, XAGE-1 mRNA can be detected by northern blotting. The expression of XAGE-1 mRNA in normal lung tissue and other normal body tissues (other than the testis) is typically too weak to be detectable by northern blotting. Thus, if the northern blot shows detectable amounts of XAGE-1, the practitioner can assume the presence of an XAGE-1-expressing cancer in the sampled tissue. If desired, the practitioner can confirm the result by quantitation of the mRNA expression. The amount of XAGE-1 mRNA in an XAGE-1 expressing cancer will typically be at least 10 times, and commonly at least 20 times, that of a normal sample of the same tissue. The diagnosis can be confirmed by knowledge of the site from which the sample was taken, histologic and morphologic features of the cells, and other routine diagnostic criteria

Xage-1 p9 or p16, immunogenic fragments of p9 or p16, nucleic acids encoding p9 or p16, or immunogenic fragments thereof can also be used ex vivo to activate cytotoxic T lymphocytes (“CTLs”) derived from a subject to attack cells of XAGE-1 expressing cancers when infused into the subject.

Xage-1 p9, p16, immunogenic fragments of p9 or p16, nucleic acids encoding these proteins, or immunogenic fragments thereof can be administered to a subject, typically in a pharmaceutically acceptable carrier, to raise or to heighten an immune response to an XAGE-1 expressing cancer. Such compositions can be administered therapeutically, in individuals who have been diagnosed as suffering from an XAGE-1 expressing cancer. In preferred embodiments, the protein or immunogenic fragments thereof are of p9 and p16 the cancers are prostate cancer, breast cancer, ovarian cancer, a lung cancer, a melanoma, a glioblastoma a T cell lymphoma or a histiocytic lymphoma Among breast cancers, cancers that do not express the estrogen receptor are preferred. In particularly preferred embodiments, the cancer detected is not a bone cancer or a muscle cancer.

Since xage-1 p16 is membrane associated, antibodies which recognize p16 can be used to target effector molecules to cells expressing p16 on the exterior surface of the cell. For example, a single-chain construct comprising the variable regions of an immunoglobulin heavy chain, a light chain, or both, can be coupled to an effector molecule such as a detectable label. The immunoconjugate can then be used to detect the presence of an xage-1 p16 expressing cell in a sample. In some embodiments, the immunoconjugate is used in vitro to detect the presence of p16-expressing cells in a sample biopsied from a patient. The presence of p16 in cells in samples taken from a site other than the testes, lung or bone marrow is indicative of the presence of an XAGE-1 expressing cancer. Normal lung cells express very small amounts of XAGE-1; accordingly, lung cells expressing significant amounts of p16 would also be indicative of an XAGE-1-expressing cancer. In particularly preferred embodiments, the cancer detected is not a bone cancer or a muscle cancer.

In other embodiments, the effector molecule of the immunoconjugate is a therapeutic agent, such as an anticancer drug, a cytotoxin, or a radioisotope, which is targeted to the cancer cells by the antibody portion of the immunoconjugate. In another group of embodiments, the immunoconjugate can be used in vitro on a culture of cells to confirm, for example, that xage-1 p16-expressing cells have been purged from the culture. In these embodiments, the effector molecule is typically a detectable label, such as a radioisotope.

In a preferred group of embodiments, the effector molecule targeted by the anti-p16 antibodies are toxins. The toxin may be a radioisotope or a chemical toxin. Suitable toxins are described in more detail below. In particularly preferred embodiments, the toxin is a Pseudomonas exotoxin A (“PE”), mutated to reduce or eliminate the non-specific binding of the toxin, or a cytotoxic fragment thereof. It should be noted that the only normal tissue found to express XAGE-1 in significant amounts are the testes. Persons of skill in the art will recognize that the testis is not essential to maintaining the life of the patient and any effect on the testis of a male patient due to the administration of an anti-xage-1 p16 immunotoxin will typically be outweighed by the therapeutic benefit to the patient of the effect of the immunotoxin on the xage-1 p16-expressing cancer.

The sections below discuss various features of xage-1 p9 and p16. The text continues with definitions used in this disclosure, with a discussion of the selection of immunogenic fragments of p9 and p16, the administration of xage-1 p9 or p16 to subjects, the formation of antibodies against xage-1 p9 or p16, detection of XAGE-1 transcript and proteins, and pharmaceutical compositions.

### Definitions

Unless defined otherwise, all technical and scientific terms used herein have the meaning commonly understood by a person skilled in the art to which this invention belongs. The following references provide a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

Reference to “XAGE-1” (that is, when printed in capital letters) refers to the XAGE-1 gene and “xage-1” (that is, when printed in lower case) refers to the protein encoded by the XAGE-1 gene.

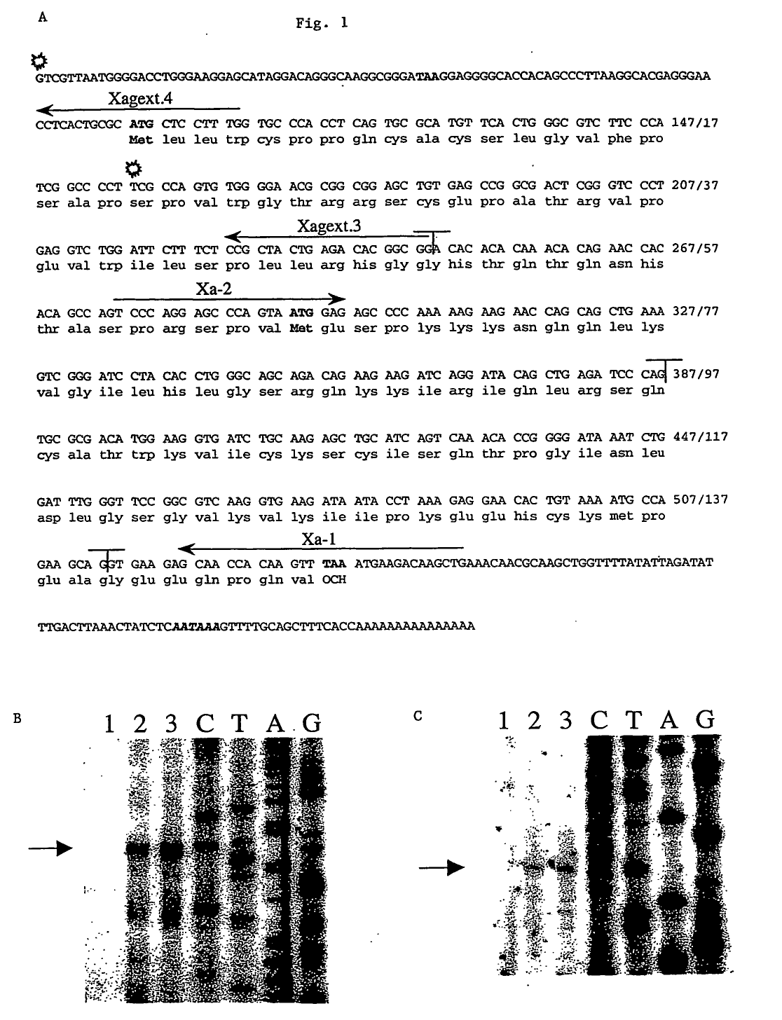

“Xage-1 p9” and “p9” refer to a protein expressed from the XAGE-1 gene having a relative molecular weight of about 9 kD. The nucleic acid sequence (SEQ ID NO:1) encoding the xage-1 9 kD protein and the amino acid sequence (SEQ ID NO:2) of xage-1 p9, are set forth in FIG. 1. The nucleic acid sequence encoding the protein starts with nucleotide 281 of the nucleotide sequence shown in FIG. 1; the amino acid sequence starts at the methionine found at position 66 of the amino acid sequence shown in that Figure.

“Xage-1 p16” and “p16” refer to a protein expressed from the XAGE-1 gene having a calculated molecular weight of about 16.3 kD. The nucleic acid sequence encoding xage-1 p16 (SEQ ID NO:3) and the amino acid sequence of xage-1 p16 (SEQ ID NO:4), are set forth in FIG. 1. The nucleic acid sequence encoding the protein starts with nucleotide 1 of the nucleotide sequence shown in FIG. 1; the amino acid sequence starts at the methionine found at position 1 of the amino acid sequence in that Figure.

As used herein, an “immunogenic fragment” of xage-1 p9 or of p16 refers to a portion of xage-1 p9 or of p16, respectively, which, when presented by a cell in the context of a molecule of the Major Histocompatibility Complex, can in a T-cell activation assay, activate a T-lymphocyte against a cell expressing XAGE-1. Typically, such fragments are 8 to 12 contiguous amino acids of xage-1 p9 or p16 in length, although longer fragments may of course also be used.

In the context of comparing one polypeptide to another, “sequence identity is determined by comparing the sequence of xage-1, as the reference sequence, to a test sequence. Typically, the two sequences are aligned for maximal or optimal alignment

A “ligand” is a compound that specifically binds to a target molecule.

A “receptor” is compound that specifically binds to a ligand.

“Cytotoxic T lymphocytes” (“CTLs”) are important in the immune response to tumor cells. CTLs recognize peptide epitopes in the context of HLA class I molecules that are expressed on the surface of almost all nucleated cells.

Tumor-specific helper T lymphocytes (“HTLs”) are also known to be important for maintaining effective antitumor immunity. Their role in antitumor immunity has been demonstrated in animal models in which these cells not only serve to provide help for induction of CTL and antibody responses, but also provide effector functions, which are mediated by direct cell contact and also by secretion of lymphokines (e.g., IFNγ and TNF-α).

“Antibody” refers to a polypeptide ligand comprising at least a light chain or heavy chain immunoglobulin variable region which specifically recognizes and binds an epitope (e.g., an antigen). This includes intact immunoglobulins and the variants and portions of them well known in the art such as, Fab′ fragments, F(ab)′₂ fragments, single chain Fv proteins (“scFv”), and disulfide stabilized Fv proteins (“dsFv”). An scFv protein is a fusion protein in which a light chain variable region of an immunoglobulin and a heavy chain variable region of an immunoglobulin are bound by a linker. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York (1997).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

“Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in METHODS IN MOLECULAR BIOLOGY, Vol. 66, Glenn E. Morris, Ed (1996).

A ligand or a receptor “specifically binds to” a compound analyte when the ligand or receptor functions in a binding reaction which is determinative of the presence of the analyte in a sample of heterogeneous compounds. Thus, the ligand or receptor binds preferentially to a particular analyte and does not bind in a significant amount to other compounds present in the sample. For example, a polynucleotide specifically binds to an analyte polynucleotide comprising a complementary sequence and an antibody specifically binds under immunoassay conditions to an antigen analyte bearing an epitope against which the antibody was raised.

“Immunoassay” refers to a method of detecting an analyte in a sample in which specificity for the analyte is conferred by the specific binding between an antibody and a ligand. This includes detecting an antibody analyte through specific binding between the antibody and a ligand. See Harlow and Lane (1988) ANTIBODICS, A LABORATORY MANUAL, Cold Spring Harbor Publications, New York, for a description of immunoassay formats and conditions that can be used to determine specific immunoreactivity.

“Vaccine” refers to an agent or composition containing an agent effective to confer a therapeutic degree of immunity on an organism while causing only very low levels of morbidity or mortality. Methods of making vaccines are, of course, useful in the study of the immune system and in preventing and treating animal or human disease.

An “immunogenic amount” is an amount effective to elicit an immune response in a subject.

A “targeting moiety” is the portion of an immunoconjugate intended to target the immunoconjugate to a cell of interest. Typically, the targeting moiety is an antibody, a scFv, a dsFv, an Fab, or an F(ab′)₂.

A “toxic moiety” is the portion of a immunotoxin which renders the immunotoxin cytotoxic to cells of interest.

A “therapeutic moiety” is the portion of an immunoconjugate intended to act as a therapeutic agent.

The term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope, where the therapeutic effect intended is, for example, the killing of a cancer cell.

A “detectable label” means, with respect to an immunoconjugate, a portion of the immunoconjugate which has a property rendering its presence detectable. For example, the immunoconjugate may be labeled with a radioactive isotope which permits cells in which the immunoconjugate is present to be detected in immunohistochemical assays.

The term “effector moiety” means the portion of an immunoconjugate intended to have an effect on a cell targeted by the targeting moiety or to identify the presence of the immunoconjugate. Thus, the effector moiety can be, for example, a therapeutic moiety, a toxin, a radiolabel, or a fluorescent label.

The term “immunoconjugate” includes reference to a covalent linkage of an effector molecule to an antibody. The effector molecule can be an immunotoxin.

The terms “effective amount” or “amount effective to” or “therapeutically effective amount” includes reference to a dosage of a therapeutic agent sufficient to produce a desired result, such as inhibiting cell protein synthesis by at least 50%, or killing the cell.

The term “toxin” includes reference to abrin, ricin, Pseudomonas exotoxin (PE), diphtheria toxin (DT), botulinum toxin, or modified toxins thereof. For example, PE and DT are highly toxic compounds that typically bring about death through liver toxicity. PE and DT, however, can be modified into a form for use as an immunotoxin by removing the native targeting component of the toxin (e.g. domain Ia of PE or the B chain of DT) and replacing it with a different targeting moiety, such as an antibody.

The term “contacting” includes reference to placement in direct physical association.

An “expression plasmid” comprises a nucleotide sequence encoding a molecule or interest, which is operably linked to a promoter.

As used herein, the term “anti-xage-1” in reference to an antibody, includes reference to an antibody which is generated against xage-1 p9 or xage-1 p16. In a particularly preferred embodiment, the antibody is generated against human xage-1 p9 or p16 synthesized by a non-primate mammal after introduction into the animal of cDNA which encodes a human xage-1 protein.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof Synthetic polypeptides can be synthesized, for example, using an automated polypeptide synthesizer. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

“Fusion protein” refers to a polypeptide formed by the joining of two or more polypeptides through a peptide bond formed by the amino terminus of one polypeptide and the carboxyl terminus of the other polypeptide. A fusion protein may is typically expressed as a single polypeptide from a nucleic acid sequence encoding the single contiguous fusion protein. However, a fusion protein can also be formed by the chemical coupling of the constituent polypeptides.

“Conservative substitution” refers to the substitution in a polypeptide of an amino acid with a functionally similar amino acid. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine M), Valine (V); and

6) Phenylalanine (F), Tyrosine (Y), Tryptophan (W).

See also, Creighton, PROTEINS, W. H. Freeman and Company, New York (1984).

Two proteins are “homologs” of each other if they exist in different species, are derived from a common genetic ancestor and share at least 70% amino acid sequence identity.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

“Nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

“Expression cassette” refers to a recombinant nucleic acid construct comprising an expression control sequence operatively linked to an expressible nucleotide sequence. An expression cassette generally comprises sufficient cis-acting elements for expression; other elements for expression can be supplied by the host cell or in vitro expression system.

“Expression vector” refers to a vector comprising an expression cassette. Expression vectors include all those known in the art, such as cosmids, plasmids (e.g., naked or contained in liposomes) and viruses that incorporate the expression cassette.

A first sequence is an “antisense sequence” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithms that are suitable for determining percent sequence identity and sequence similarity are the BLAST and the BLAST 2.0 algorithm, which are described in Altschul et al., J. Mol. Biol. 215:403-410 (1990) and Altschul et al., Nucleic Acids Res. 25:3389-3402 (1977)). Software for performing BLAST analyses is publicly available through the National Center for Biotechnology Information (http://www.nobi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915 (1989)).

“Stringent hybridization conditions” refers to 50% formamide, 5×SSC and 1% SDS incubated at 42° C. or 5×SSC and 1% SDS incubated at 65° C., with a wash in 0.2×SSC and 0.1% SDS at 65° C.

“Naturally-occurring” as applied to an object refers to the fact that the object can be found in nature. For example, an amino acid or nucleotide sequence that is present in an organism (including viruses) that can be isolated from a source in nature and which has not been intentionally modified by man in the laboratory is naturally-occurring.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

“Pharmaceutical composition” refers to a composition suitable for pharmaceutical use in a mammal. A pharmaceutical composition comprises a pharmacologically effective amount of an active agent and a pharmaceutically acceptable carrier.

“Pharmacologically effective amount” refers to an amount of an agent effective to produce the intended pharmacological result.

“Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in REMINGTON'S PHARMACEUTICAL SCIENCES, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound for pharmaceutical use including, e.g., metal salts (sodium, potassium, magnesium, calcium, etc.) and salts of ammonia or organic amines.

A “subject” of diagnosis or treatment is a human or non-human mammal.

“Administration” of a composition refers to introducing the composition into the subject by a chosen route of administration. For example, if the chosen route is intravenous, the composition is administered by introducing the composition into a vein of the subject.

“Treatment” refers to prophylactic treatment or therapeutic treatment.

A “prophylactic” treatment is a treatment administered to a subject who does not exhibit signs of a disease or exhibits only early signs for the purpose of decreasing the risk of developing pathology.

A “therapeutic” treatment is a treatment administered to a subject who exhibits signs of pathology for the purpose of diminishing or eliminating those signs.

“Diagnostic” means identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is I minus the false positive rate, where the false positive rate is defined as the proportion of those without the disease who test positive. While a particular diagnostic method may not provide a definitive diagnosis of a condition, it suffices if the method provides a positive indication that aids in diagnosis.

“Prognostic” means predicting the probable development (e.g., severity) of a pathologic condition.

### Proteins Sythesized from XAGE-1

This invention provides isolated, recombinant proteins synthesized from XAGE-1. Two proteins are expressed from XAGE-1. First, in transfection experiments, cells transfected with XAGE-1 synthesize a 9 kD protein which is termed herein xage-1 p9. With reference to FIG. 1, the sequence of p9 is shown commencing with the methionine residue found at position 66 of the amino acid sequence. Second, a protein with a putative molecular weight of 16 kD can be synthesized from XAGE-1. The sequence of this protein is also shown in FIG. 1, commencing with the methionine found at position 1 of the amino acid sequence. The nucleotide sequence encoding the proteins is set forth above the respective amino acid sequences. Because of the degeneracy of the genetic code, persons of skill will recognize that numerous other nucleotide sequences could encode the same amino acid sequences.

In certain embodiments, this invention provides polypeptides comprising an epitope comprising at least 5 to at least 15 consecutive amino acids from p9 or from p16. Such proteins bind to antibodies raised against full-length p9 or p16, respectively. Since p16 comprehends the amino acid sequence of p9, but includes an additional amino acid sequence at the N-terminal end, (that is, amino acids 1-65), it is expected that antibodies raised against epitopes found on p9 will bind to p16, but that antibodies raised to p16 may or may not bind to p9 depending on whether the epitope is found in- amino acids 1-65 of p16 (in which case the antibody will not recognize p9 unless there is an equivalent sequence in p9) or the epitope occurs in amino acids 66-146 of p16, in which case the antibody will bind p9.

In other embodiments, this invention provides fusion proteins comprising a first and second polypeptide moiety in which one of the protein moieties comprises an amino acid sequence of at least 5 amino acids identifying an epitope of xage-1 p9 or p16. In one embodiment the xage-1 moiety is all or substantially of p9 or p16. The other moiety can be, e.g., an immunogenic protein. Such fusions also are useful to evoke an immune response against xage-1 p9 or p16, respectively. In preferred embodiments, the protein is p9, and the immune response is raised against cells expressing p9.

In other embodiments, this invention provides xage-1 p9-like peptides (“p9 analogs”) whose amino acid sequences are at least 90% identical to p9 (although they may have 91%, 92%, 93%, 94%, 95%, or even higher sequence identity to p9) and which are specifically bound by antibodies which specifically bind to xage-1 p9. In preferred embodiments this invention provides xage-1 p9-like peptides (also sometimes referred to herein as “p9-analogs”) whose amino acid sequences are at least 90% identical to p9 (although they may have 91%, 92%, 93%, 94%, 95%, or even higher sequence identity to p9) and which activate T-lymphocytes to cells which express xage-1 p9.

Similarly, in some embodiments, this invention provides xage-1 p16-like peptides (“p16 analogs”) whose amino acid sequences are at least 90% identical to p16 (although they may have 91%, 92%, 93%, 94%, 95%, or even higher sequence identity to p16) and which are specifically bound by antibodies which specifically bind to xage-1 p16. In preferred embodiments this invention provides xage-1 p16-like peptides (also sometimes referred to herein as “p16-analogs”) whose amino acid sequences are at least 90% identical to p16 (although they may have 91%, 92%, 93%, 94%, 95%, or even higher sequence identity to p16) and which activate T-lymphocytes to cells which express xage-1 p16.

In another embodiment the polypeptide comprises an epitope that binds an MHC molecule, e.g., an HLA molecule or a DR molecule. These molecules bind polypeptides having the correct anchor amino acids separated by about eight or nine amino acids. These peptides can be identified by inspection of the amino acid sequence of p9 and by knowledge of the MHC binding motifs, well known in the art.

Xage-1 p9, p16, immunogenic fragments of these proteins, and p9 and p16 analogs can be synthesized recombinantly. Imunogenic fragments of p9 and p16 and the full length proteins can also be chemically synthesized by standard methods. If desired, polypeptides can also be chemically synthesized by emerging technologies. One such process is described in W. Lu et al., Federation of European Biochenzical Societies Letters. 429:31-35 (1998).

### XAGE-1 Nucleic Acids

In one aspect this invention provides isolated, recombinant nucleic acid molecules comprising nucleotide sequences encoding the xage-1 p9 and p16 proteins (see, e.g., FIG. 1). The nucleic acids are useful for expressing p9 and p16, which can then be used, for example, to raise antibodies for diagnostic purposes. As noted, XAGE-1 is translated as two proteins which have alternative start codons. The nucleic acid sequence encoding p16 (SEQ ID NO:3) commences with the first nucleotide shown in FIG. 1; the nucleic acid sequence encoding p9 (SEQ ID NO:1) commences with the first nucleotide of the codon encoding the methionine at position 66 of the amino acid sequence shown in FIG. 1.

The practitioner can use these sequences to prepare PCR primers for isolating nucleotide sequences of the invention. Exemplary primers are set forth in the Examples, below. The positions in the XAGE-1 nucleotide sequence to which the primers hybridize are set forth in FIG. 1. The sequences encoding p9 and p16 can be modified to engineer nucleic acids encoding related molecules of this invention using well known techniques.

A nucleic acid comprising sequences of the invention can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the p9 or the p16 protein can be isolated by polymerase chain reaction of cDNA using primers based on the DNA sequence of the molecule.

A wide variety of cloning and in vitro amplification methodologies are well-known to persons skilled in the art. PCR methods are described in, for example, U.S. Pat. No. 4,683,195; Mullis et al. (1987) Cold Spring Harbor Symp. Quant. Biol. 51:263; and Erlich, ed., PCR TECHNOLOGY, (Stockton Press, New York, 1989). Polynucleotides also can be isolated by screening genomic or cDNA libraries with probes selected from the sequences of the desired polynucleotide under stringent hybridization conditions.

Engineered versions of the nucleic acids can be made by site-specific mutagenesis of other polynucleotides encoding the proteins, or by random mutagenesis caused by increasing the error rate of PCR of the original polynucleotide with 0.1 mM MnCl₂ and unbalanced nucleotide concentrations.

### A. Expression vectors

The invention also provides expression vectors for expressing p9 and p16. Construction of an exemplary expression vector is discussed in the Examples, below. Expression vectors can be adapted for function in prokaryotes or eukaryotes by inclusion of appropriate promoters, replication sequences, markers, etc. for transcription and translation of mRNA. The construction of expression vectors and the expression of genes in transfected cells involves the use of molecular cloning techniques also well known in the art. Sambrook et al., MOLECULAR CLONING—A LABORATORY MANUAL, Cold Spring Harbor Laboratory, Cold Spring Harbor, N.Y., (1989) and CURRENT PROTOCOLS IN MOLECULAR BIOLOGY, F. M. Ausubel et al., eds., (Current Protocols, Greene Publishing Associates, Inc. and John Wiley & Sons, Inc.) (“Ausubel”). Useful promoters for such purposes include a metallothionein promoter, a constitutive adenovirus major late promoter, a dexamethasone-inducible MMTV promoter, a SV40 promoter, a MRP polIII promoter, a constitutive MPSV promoter, a tetracycline-inducible CMV promoter (such as the human immediate-early CMV promoter), and a constitutive CMV promoter. A plasmid useful for gene therapy can comprise other functional elements, such as selectable markers, identification regions, and other genes.

Expression vectors useful in this invention depend on their intended use. Such expression vectors must, of course, contain expression and replication signals compatible with the host cell. Expression vectors useful for expressing bioactive conjugates include viral vectors such as retroviruses, adenoviruses and adeno-associated viruses, plasmid vectors, cosmids, and the like. Viral and plasmid vectors are preferred for transfecting mammalian cells. The expression vector pcDNA3 (Invitrogen, San Diego, Calif.), in which the expression control sequence comprises the CMV promoter, provides good rates of transfection and expression. Adeno-associated viral vectors are useful in the gene therapy methods of this invention.

A variety of means are available for delivering polynucleotides to cells including, for example, direct uptake of the molecule by a cell from solution, facilitated uptake through lipofection (e.g., liposomes or immunoliposomes), particle-mediated transfection, and intracellular expression from an expression cassette having an expression control sequence operably linked to a nucleotide sequence that encodes the inhibitory polynucleotide. See also U.S. Pat. No. 5,272,065 (Inouye et al.); METHODS IN ENZYMOLOGY, vol. 185, Academic Press, Inc., San Diego, Calif. (D. V. Goeddel, ed.) (1990) or M. Krieger, GENE TRANSFER AND EXPRESSION—A LABORATORY MANUAL, Stockton Press, New York, N.Y., (1990). Recombinant DNA expression plasmids can also be used to prepare the polynucleotides of the invention for delivery by means other than by gene therapy, although it may be more economical to make short oligonucleotides by in vitro chemical synthesis.

The construct can also contain a tag to simplify isolation of the protein. For example, a polyhistidine tag of, e.g. six histidine residues, can be incorporated at the amino terminal end of the protein. The polyhistidine tag allows convenient isolation of the protein in a single step by nickel-chelate chromatography.

### B. Recombinant Cells

The invention also provides recombinant cells comprising an expression vector for expression of the nucleotide sequences of this invention (“host cells”). Host cells can be selected for high levels of expression in order to purify the protein. The cells can be prokaryotic cells, such as E. coli, or eukaryotic cells. Useful eukaryotic cells include yeast and mammalian cells. The cell can be, e.g., a recombinant cell in culture or a cell in vivo.

Cells expressing p9 or p16 are useful for active or passive immunization of subjects against cells expressing these peptides. In certain embodiments, the cells are bacterial cells. In one version of active immunization, recombinant cells are autologous cells of the subject that can present the polypeptides in association with HLA molecules. For example, antigen presenting cells are useful for this purpose. In this case, it is preferable to use “autologous cells,” that is, cells derived from the subject. Such cells are MHC compatible. The p9- or p16-encoding nucleotide sequence should be placed under the control of a constitutive promoter in such cells because one goal is to express the polypeptides in high density on the cell surface, preferably more densely than they are expressed in healthy testis cells.

### Methods of Eliciting a Cell-Mediated Immune Response Against Cells Expressing XAGE-1

XAGE-1 is expressed by cells of a number of cancers, including cancers of the prostate, breast, ovaries, lung and pancreas, in addition to some muscle and bone cancers. Therefore, XAGE-1 can be used as a target of intervention in inhibiting the growth of cells of these cancers which express XAGE-1, as well as a marker for cancer cells that have metastasized from these cancers. This invention provides methods of treating these cancers with immunotherapy. The methods involve immunizing a subject against p9 or p16, or both, thereby eliciting a cell-mediated immune response against cells expressing these proteins. Immunization can be active or passive. In active immunization, the immune response is elicited in the subject in vivo. In passive immunization, T_{C} cells activated against the polypeptide are cultured in vitro and administered to the subject. Such methods may be expected to result in the destruction of healthy testis tissue that expresses XAGE-1. However, the testes are not an essential organ. Loss of the testes must be counterbalanced against the chance for loss of the subject's life from the cancer, and the testes may, indeed, be surgically removed prior to institution of immunotherapy.

The immunizing agent can be of full-length p9 or p16, a peptide comprising an antigenic determinant of p9 or p16, e.g., an immunogenic fragment of p9, or a protein or peptide that is substantially identical to p9 or p16. In preferred embodiments, the immuning agent is full-length p9, an immunogenic fragment thereof, or a protein or peptide that is substantially identical to p9 (that is, which has 90% or more sequence identity to p9 and preferably about 95% or more sequence identity). When one is attempting to elicit a cell-mediated immune response against XAGE-1, preferred peptides comprising antigenic determinants are those peptides bearing a binding motif for an HLA molecule of the subject. These motifs are well known in the art. For example, HLA-A2 is a common allele in the human population. The binding motif for this molecule includes polypeptides with 9 or 10 amino acids having leucine or methionine in the second position and valine or leucine in the last positions.

Based on the polypeptide sequence of p9 and p16, one can identify amino acid sequences bearing motifs for any particular HLA molecule. Peptides comprising these motifs can be prepared by any of the typical methods (e.g., recombinantly, chemically, etc.). Because p9 and p16 are self proteins, the preferred amino acid sequences bearing HLA binding motifs are those that encode subdominant or cryptic epitopes. Those epitopes can be identified by a lower comparative binding affinity for the HLA molecule with respect to other epitopes in the molecule or compared with other molecules that bind to the HLA molecule.

Polypeptides that comprise an amino acid sequence from p9 or p16 that, in turn, comprise an HLA binding motif also are useful for eliciting an immune response. This is because, in part, such proteins will be processed by the cell into a peptide that can bind to the HLA molecule and that have a p9 or p16 epitope.

A complex of an HLA molecule and a peptidic antigen acts as the ligand recognized by HLA-restricted T cells (Buus, S. et al., Cell 47:1071 (1986); Babbitt, B. P. et al., Nature 317:359 (1985); Townsend, A. and Bodmer, H., Annu. Rev. Immunol. 7:601, 1989; Germain, R. N., Annu. Rev. Immunol. 11:403 (1993)). Through the study of single amino acid substituted antigen analogs and the sequencing of endogenously bound, naturally processed peptides, critical residues that correspond to motifs required for specific binding to HLA antigen molecules have been identified (see, e.g., Southwood, et al., J. Immunol. 160:3363 (1998); Rammensee, et al., Immunogenetics 41:178 (1995); Rammensee et al., Sette, A. and Sidney, J. Curr. Opin. Immunol. 10:478 (1998); Engelhard, V. H., Curr. Opin. Immunol. 6:13 (1994); Sette, A. and Grey, H. M., Curr. Opin. Immunol. 4:79, (1992)).

Furthermore, x-ray crystallographic analysis of HLA-peptide complexes has revealed pockets within the peptide binding cleft of HLA molecules which accommodate, in an allele-specific mode, residues borne by peptide ligands; these residues in turn determine the HLA binding capacity of the peptides in which they are present. (See, e.g., Madden, D. R. Annu. Rev. Immunol. 13:587,1995; Smith, et al., Immunity 4:203,1996; Fremont et al., Immunity 8:305, 1998; Stem et al., Structure 2:245, 1994; Jones, E. Y. Curr. Opin. Immunol. 9:75, 1997; Brown, J. H. et al., Nature 364:33, 1993.)

Accordingly, the definition of class I and class II allele-specific HLA binding motifs, or class I or class II supermotifs allows identification of regions within p9 or p16 that have the potential of binding particular HLA molecules.

Molecules with high levels of sequence identity to p9 or p16 are also useful to elicit an immune response. Such molecules can be recognized as “foreign” to the immune system, yet generate antibodies or CTLs that cross react with p9 or p16. Analogs of p9 whose amino acid sequences are at least 90% identical to p9 (although they may have 91%, 92%, 93%, 94%, 95%, or even higher sequence identity to p9) and which are specifically bound by antibodies which specifically bind to p9 may be used. Further useful in this regard are p9 analogs, that is, peptides whose amino acid sequences are at least 90% identical to p9 (although they may have 91%, 92%, 93%, 94%, 95%, or even higher sequence identity to p9) and which activate T-lymphocytes to cells which express p9. Similarly, analogs of p16 whose amino acid sequences are at least 90% identical to p16 (although they may have 91%, 92%, 93%, 94%, 95%, or even higher sequence identity to p16) and which are specifically bound by antibodies which specifically bind to p9 may be used. Further useful in this regard are p16 analogs, that is, peptides whose amino acid sequences are at least 90% identical to p16 (although they may have 91%, 92%, 93%, 94%, 95%, or even higher sequence identity to p16) and which activate T-lymphocytes to cells which express p16.

Another molecule that is substantially homologous to a p9 or p16 antigenic determinant can be made by modifying the sequence of a natural p9 or p16 epitope so that it binds with greater affinity for the HLA molecule.

One method of identifying genes encoding antigenic determinants is as follows: TILs from a subject with metastatic cancer are grown and tested for the ability to recognize the autologous cancer in vitro. These TILs are administered to the subject to identify the ones that result in tumor regression. The TILs are used to screen expression libraries for genes that express epitopes recognized by the TILs. Subjects then are immunized with these genes. Altematively, lymphocytes are sensitized in vitro against antigens encoded by these genes. Then the sensitized lymphocytes are adoptively transferred into subjects and tested for their ability to cause tumor regression. Rosenberg, et al., Immunol. Today 1997 18:175 (1997).

The application of these molecules is now described. These methods are also described in Rosenberg et al., supra, and Restifo et al., Oncology 11:50 (1999).

One method of involing an immune response involves immunizing the subject with a polypeptide comprising an antigenic determinant from p9 or p16, either alone or, more preferably, combined with an adjuvant, such as Freund's incomplete adjuvant, lipids or liposomes, gp96, Hsp70 or Hsp90. The polypeptide can be p9 or p16, an antigenic fragment of p9 or p16, a fusion protein comprising the antigenic determinant, or a peptide comprising a sequence substantially identical to such an antigenic determinant.

Another method involves pulsing a polypeptide comprising an epitope from p9 or p16 onto antigen presenting cells and administering the cells to the subject.

In another method, a recombinant virus containing a nucleic acid sequence encoding a polypeptide comprising an antigenic determinant from p9 or p16 in an expression cassette is administered to the subject. The virus optionally also can encode cytolines (e.g., IL-2), a costimulatory molecule or other genes that enhance the immune response. The virus can be, for example, adenovirus, fowlpox virus or vaccinia virus. Upon infection, the infected cells will express the p9 or p16 peptide and express the antigenic determinant on the cell surface in combination with the HLA molecule which binds peptides having the same motif as the antigenic determinant. These cells will then stimulate the activation of CTLs that recognize the presented antigen, resulting in destruction of cancer cells that also bear the determinant.

In another method, the subject is immunized with naked DNA encoding a polypeptide comprising an antigenic determinant from p9 or p16 by, e.g., intramuscular, biolistic injection or linked to lipids. Such methods have been shown to result in the stimulation of a cell-mediated response against cells that express the encoded polypeptide.

In another method, recombinant bacteria that express the epitope, such as Bacillus Calmette-Guerin (BCG), Salmonella or Listeria, optionally also encoding cytokines, costimulatory molecules or other genes to enhance the immune response, are administered to the subject.

In another method, cells expressing the antigen are administered to the subject. This includes, for example, dendritic cells pulsed with p9 or p16 epitopes, cells transfected with polypeptides comprising p9 or p16 antigenic determinants, HLA and B7 genes. The multiple transfection results in the production of several components necessary for presenting the antigenic determinant on the cell surface. In one embodiment, the molecule is a fusion protein in which the polypeptide bearing the antigenic determinant is fused to an HLA molecule (usually through a linker) so as to improve binding of the peptide to the HLA molecule. In one embodiment, the cell is an antigen presenting cell. Preferably, the cells are eukaryotic cells, more preferably, mammalian cells, more preferably, human cells, more preferably autologous human cells derived from the subject.

In another method, antigen presenting cells (APCs) are pulsed or co-incubated with peptides comprising an epitope from p9 or p16 in vitro. These cells are used to sensitize CD8 cells, such as tumor infiltrating lymphocytes from prostate cancer tumors or peripheral blood lymphocytes. The TILs or PBLs preferably are from the subject. However, they should at least be MHC Class-I restricted to the HLA types the subject possesses. The sensitized cells are then administered to the subject.

In a supplemental method, any of these immunotherapies is augmented by administering a cytokine, such as IL-2, IL-3, IL-6, IL-10, IL-12, IL-15, GM-CSF, interferons.

In addition to the methods for evaluating immunogenicity of peptides set forth above, immunogenicity can also be evaluated by: evaluation of primary T cell cultures from normal individuals (see, e.g., Wentworth, P. A. et al., Mol. Immunol. 32:603, 1995; Celis, E. et al., Proc. Natl. Acad. Sci. USA 91:2105, 1994; Tsai, V. et al., J. Immunol. 158:1796, 1997; Kawashima, I. et al., Human Immunol. 59:1, 1998); by immunization of HLA transgenic mice (see, e.g., Wentworth, P. A. et al., J. Immunol. 26:97, 1996; Wentworth, P. A. et al., Int. Immunol. 8:651, 1996; Alexander, J. et al., J. Immunol. 159:4753, 1997), and by demonstration of recall T cell responses from patients who have been effectively vaccinated or who have a tumor; (see, e.g., Rehermann, B. et al., J. Exp. Med. 181:1047, 1995; Doolan, D. L. et al., Immunity 7:97, 1997; Bertoni, R et al., J. Clin. Invest. 100:503, 1997; Threlkeld, S. C. et al., J. Immunol. 159:1648, 1997; Diepolder, H. M. et al., J. Virol. 71:6011, 1997).

In choosing CTL-inducing peptides of interest for vaccine compositions, peptides with higher binding affinity for class I HLA molecules are generally preferable. Peptide binding is assessed by testing the ability of a candidate peptide to bind to a purified HLA molecule in vitro.

To ensure that a p9 or p16 analog when used as a vaccine, actually elicits a CTL response to p9 or p16 in vivo (or, in the case of class II epitopes, elicits helper T cells that cross-react with the wild type peptides), the p9 or p16 analog may be used to immunize T cells in vitro from individuals of the appropriate HLA allele. Thereafter, the immunized cells' capacity to induce lysis of p9- or p16-sensitized target cells is evaluated.

More generally, peptides from p9 or p16 or an analog thereof (a “peptide of the invention”) can be synthesized and tested for their ability to bind to HLA proteins and to activate HTL or CTL responses, or both.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations.

PBMCs may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

A method which allows direct quantification of antigen-specific T cells is staining with Fluorescein-labeled HLA tetrameric complexes (Altman et al., Proc. Natl. Acad. Sci. USA 90:10330 (1993); Altman et al., Science 274:94 (1996)). Alternatively, staining for intracellular lymphokines, interferon-γ release assays or ELISPOT assays, can be used to evaluate T-cell responses.

HTL activation may be assessed using such techniques known to those in the art such as T cell proliferation and secretion of lymphokines, e.g. IL-2 (see, e.g. Alexander et al., Immunity 1:751-761 (1994)).

### Antibodies Against p9 and p16

The anti-p9 or p16 antibodies generated in the present invention can be linked to effector molecules (EM) through the EM carboxyl terminus, the EM amino terminus, through an interior amino acid residue of the EM such as cysteine, or any combination thereof Similarly, the EM can be linked directly to the heavy, light, Fc (constant region) or framework regions of the antibody. Linkage can occur through the antibody's amino or carboxyl termini, or through an interior amino acid residue. Further, multiple EM molecules (e.g., any one of from 2-10) can be linked to the anti-p9 or p16 antibody and/or multiple antibodies (e.g., any one of from 2-5) can be linked to an EM. The antibodies used in a multivalent immunoconjugate composition of the present invention can be directed to the same or different p9 or p 16 epitopes.

In preferred embodiments of the present invention, the anti-p9 or p16 antibody is a recombinant antibody such as a scFv or a disulfide stabilized Fv antibody. Fv antibodies are typically about 25 kDa and contain a complete antigen-binding site with 3 CDRs per heavy and light chain. If the V_{H} and the V_{L} chain are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker.

In a particularly preferred embodiment, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. In a more preferred embodiment, the scFv is recombinantly produced. One of skill will realize that conservative variants of the antibodies of the instant invention can be made. Such conservative variants employed in scFv fragments will retain critical amino acid residues necessary for correct folding and stabilizing between the V_{H} and the V_{L} regions.

In some embodiments of the present invention, the scFv antibody is directly linked to the EM through the light chain. However, scFv antibodies can be linked to the EM via its amino or carboxyl terminus.

While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston, et al., Proc. Nat'l Acad. Sci. USA 8:5879 (1988); Bird, et al., Science 242:4236 (1988); Glockshuber, et al., Biochemistry 29:1362 (1990); U.S. Pat. Nos.4,946,778, 5,132,405 and Stemmer, et al., Biotechniques 14:256-265 (1993), all incorporated herein by reference. Generally the peptide linker will have no specific biological activity other than to join the regions or to preserve some minimum distance or other spatial relationship between them. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Ser, preferably 2, 3, 4, 5, or 6 such sequences. However, it is to be appreciated that some amino acid substitutions within the linker can be made. For example, a valine can be substituted for a glycine.

### A. Antibody Production

Methods of producing polyclonal antibodies are known to those of skill in the art. In brief, an immunogen, preferably isolated p9 or p16 or extracellular p9 or p16 epitopes are mixed with an adjuvant and animals are immunized with the mixture. When appropriately high titers of antibody to the immunogen are obtained, blood is collected from the animal and antisera are prepared. If desired, further fractionation of the antisera to enrich for antibodies reactive to the polypeptide is performed. See, e.g., Coligan, CURRENT PROTOCOLS IN IMMUNOLOGY, Wiley/Greene, New York (1991); and Harlow & Lane, supra, which are incorporated herein by reference.

A number of immunogens can be used to produce antibodies that specifically bind p9 or p16. Full-length p9 or p16 is a suitable immunogen. Typically, the immunogen of interest is a peptide of at least about 3 amino acids, more typically the peptide is at least 5 amino acids in length, preferably, the fragment is at least 10 amino acids in length and more preferably the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form.

Monoclonal antibodies may be obtained by various techniques familiar to those skilled in the art. Description of techniques for preparing such monoclonal antibodies may be found in, e.g., Stites, et al. (eds.) BASIC AND CLINICAL IMMUNOLOGY (4TH ED.), Lange Medical Publications, Los Altos, Calif., and references cited therein; Harlow & Lane, supra; Goding, MONOCLONAL ANTIBODIES: PRINCIPLES AND PRACTICE (2D ED.), Academic Press, New York, N.Y. (1986); Kohler & Milstein, Nature 256:495-497 (1975); and particularly (Chowdhury, P. S., et al., Mol. Immunol. 34:9 (1997)), which discusses one method of generating monoclonal antibodies.

It is preferred that monoclonal antibodies are made by immunizing an animal with the target antigen or with nucleic acid sequence that encodes the desired immunogen, such as p9 or p16. Immunization with non-replicating transcription units that encode a heterologous proteins elicits antigen specific immune responses. After translation into the foreign protein, the protein is processed and presented to the immune system like other cellular proteins. Because it is foreign, an immune response is mounted against the protein and peptide epitopes that are derived from it (Donnelly, et al., J. Immunol. Methods 176:145-152 (1994); and Boyer, et al., J. Med. Prinatol. 25:242-250 (1996)). This technique has two significant advantages over protein-based immunization. One is that it does not require the purification of the protein, which at best, is time consuming and in cases of many membrane proteins, is very difficult. A second advantage is that since the immunogen is synthesized in a mammalian host, it undergoes proper post-translational modifications and folds into the native structure.

To immunize with p9- or p16-coding DNA, p9- or p16-coding cDNA is introduced into a plasmid so that transcription of the coding sequence is under the control of a promoter such as the CMV promoter. The plasmid is then injected into an animal, either subcutaneously, intradermally, intraperitoneally, etc. As a result, the p9 or p16 cDNA is transcribed in the animal into mRNA, p9 or p16 is translated from the mRNA, the translated protein undergoes proper post-translational modifications and is expressed on the surface of cells which synthesized p9 or p16. The animal raises antibodies to p9 or p16 and the sera is monitored for antibody titer.

Optionally, in addition to the coding region and regulatory elements, the plasmid carries an ampicillin resistance (Amp) gene. The Amp gene is known to have immunostimulatory sequences for Th1 responses necessary for increased antibody production (Sato, et al., Science 273:352-354 (1996)).

As described above, in preferred embodiments, the monoclonal antibody is a scFv. Methods of making scFv antibodies have been described. See, Huse, et al., supra; Ward, et al. Nature 341:544-546 (1989); and Vaughan, et al., supra. In brief, mRNA from B-cells is isolated and cDNA is prepared. The cDNA is amplified by well known techniques, such as PCR, with primers specific for the variable regions of heavy and light chains of immunoglobulins. The PCR products are purified by, for example, agarose gel electrophoresis, and the nucleic acid sequences are joined. If a linker peptide is desired, nucleic acid sequences that encode the peptide are inserted between the heavy and light chain nucleic acid sequences. The sequences can be joined by techniques known in the art, such as blunt end ligation, insertion of restriction sites at the ends of the PCR products or by splicing by overlap extension (Chowdhury, et al., Mol. Immunol. 34:9 (1997)). After amplification, the nucleic acid which encodes the scFv is inserted into a vector, again by techniques well known in the art. Preferably, the vector is capable of replicating in prokaryotes and of being expressed in both eukaryotes and prokaryotes.

In a preferred embodiment, scFv are chosen through a phage display library. The procedure described above for synthesizing scFv is followed. After amplification by PCR, the scFv nucleic acid sequences are fused in frame with gene III (gIII) which encodes the minor surface protein gIIIp of the filamentous phage (Marks, et al., J. Biol. Chem. 267:16007-16010 (1992); Marks, et al., Behring Inst. Mitt. 91:6-12 (1992); and Brinkmann, et al., J. Immunol. Methods 182:41-50 (1995)). The phage express the resulting fusion protein on their surface. Since the proteins on the surface of the phage are functional, phage bearing p9- or p16-binding antibodies can be separated from non-binding or lower affinity phage by panning or antigen affinity chromatography (McCafferty, et al., Nature 348:552-554 (1990)).

In a preferred embodiment, scFv that specifically bind to p9 or p16 are found by panning. Panning is done by coating a solid surface with p9 or p16 and incubating the phage on the surface for a suitable time under suitable conditions. The unbound phage are washed off the solid surface and the bound phage are eluted. Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to p9 or p16 coated plates are expanded in E. coli and subjected to another round of panning. In this way, an enrichment of 2000-fold occurs in 3 rounds of panning. Thus, even when enrichment in each round is low, multiple rounds of panning will lead to the isolation of rare phage and the genetic material contained within which encodes the sequence of the highest affinity antibody. The physical link between genotype and phenotype provided by phage display makes it possible to test every member of a cDNA library for binding to antigen, even with large libraries of clones.

### B. Binding Affinity of Antibodies

Binding affinity for a target antigen is typically measured or determined by standard antibody-antigen assays, such as competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

### C. Immunoassays

The antibodies can be detected and/or quantified using any of a number of well recognized immunological binding assays (see, e.g., U.S. Pat. Nos. 4,366,241; 4,376,110; 4,517,288; and 4,837,168). For a review of the general immunoassays, see also METHODS IN CELL BIOLOGY, VOL. 37, Asai, ed. Academic Press, Inc. New York (1993); BASIC AND CLINICAL IMMUNOLOGY 7TH EDITION, Stites & Terr, eds. (1991). Immunological binding assays (or immunoassays) typically utilize a ligand (e.g., p9 or p 16) to specifically bind to and often immobilize an antibody. The antibodies employed in immunoassays of the present invention are discussed in greater detail supra.

Immunoassays also often utilize a labeling agent to specifically bind to and label the binding complex formed by the ligand and the antibody. The labeling agent may itself be one of the moieties comprising the antibody/analyte complex, i.e., the anti-p9 or p16 antibody. Alternatively, the labeling agent may be a third moiety, such as another antibody, that specifically binds to the antibody/ p9 or p16 protein complex.

In one aspect, a competitive assay is contemplated wherein the labeling agent is a second anti-p9 or p16 antibody bearing a label. The two antibodies then compete for binding to the immobilized p9 or p16. Alternatively, in a non-competitive format, the anti-p9 or p16 antibody lacks a label, but a second antibody specific to antibodies of the species from which the anti-p9 or p16 antibody is derived, e.g., murine, and which binds the anti-p9 or p16 antibody, is labeled.

Other proteins capable of specifically binding immunoglobulin constant regions, such as Protein A or Protein G may also be used as the label agent. These proteins are normal constituents of the cell walls of streptococcal bacteria They exhibit a strong non-immunogenic reactivity with immunoglobulin constant regions from a variety of species (see, generally Kronval, et al., J. Immunol. 111:1401-1406 (1973); and Akerstrom, et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antibody, volume of solution, concentrations, and the like. Usually, the assays will be carried out at ambient temperature, although they can be conducted over a range of temperatures, such as 10° C. to 40° C.

While the details of the immunoassays of the present invention may vary with the particular format employed, the method of detecting anti-p9 or p16 antibodies in a sample containing the antibodies generally comprises the steps of contacting the sample with an antibody which specifically reacts, under immunologically reactive conditions, to the p9 or p16/antibody complex.

### Production of Immunoconjugates

Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents may include various drugs such as vinblastine, daunomycin and the like, cytotoxins such as native or modified Pseudomonas exotoxin or Diphtheria toxin, encapsulating agents, (e.g., liposomes) which themselves contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell and the biological effect is desired to evoke. Thus, for example, the therapeutic agent may be a cytotoxin which is used to bring about the death of a particular target cell. Conversely, where it is merely desired to invoke a non-lethal biological response, the therapeutic agent may be conjugated to a non-lethal pharmacological agent or a liposome containing a nonlethal pharmacological agent.

With the therapeutic agents and antibodies herein provided, one of skill can readily construct a variety of clones containing functionally equivalent nucleic acids, such as nucleic acids which differ in sequence but which encode the same EM or antibody sequence. Thus, the present invention provides nucleic acids encoding antibodies and conjugates and fusion proteins thereof

### A. Recombinant Methods

Nucleic acid sequences encoding the chimeric molecules of the present invention can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook, et al., supra, Berger and Kimmel (eds.), supra, and Ausubel, supra. Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding native EM or anti-p9 or p16 antibodies can be modified to form the EM, antibodies, or immunoconjugates of the present invention. Modification by site-directed mutagenesis is well known in the art. Nucleic acids encoding EM or anti-p9 or p16 antibodies can be amplified by in vitro methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, immunoconjugates are prepared by inserting the cDNA which encodes an anti-p9 or p16 scFv antibody into a vector which comprises the cDNA encoding the EM. The insertion is made so that the scFv and the EM are read in frame, that is in one continuous polypeptide which contains a functional Fv region and a functional EM region. In a particularly preferred embodiment, cDNA encoding a diphtheria toxin fragment is ligated to a scFv so that the toxin is located at the carboxyl terminus of the scFv. In a most preferred embodiment, cDNA encoding PE is ligated to a scFv so that the toxin is located at the amino terminus of the scFv.

Once the nucleic acids encoding an EM, anti-p9 or p16 antibody, or an immunoconjugate of the present invention are isolated and cloned, one may express the desired protein in a recombinantly engineered cell such as bacteria, plant, yeast, insect and mammalian cells as discussed above in connection with the discussion of expression vectors encoding p9 or p16. It is expected that those of skill in the art are knowledgeable in the numerous expression systems available for expression of proteins including E. coli other bacterial hosts, yeast, and various higher eukaryotic cells such as the COS, CHO, HeLa and myeloma cell lines. No attempt to describe in detail the various methods known for the expression of proteins in prokaryotes or eukaryotes will be made.

One of skill would recognize that modifications can be made to a nucleic acid encoding a polypeptide of the present invention (i.e., anti-p9 or p16 antibody, PE, or an immunoconjugate formed from their combination) without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation site, additional amino acids placed on either terminus to create conveniently located restriction sites, or additional amino acids (such as poly His) to aid in purification steps.

In addition to recombinant methods, the immunoconjugates, EM, and antibodies of the present invention can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A. pp. 3-284; Merrifield, et al. J. Am. Chem. Soc. 85:2149-2156 (1963), and Stewart, et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill.

### B. Purification

Once expressed, the recombinant immunoconjugates, antibodies, and/or effector molecules of the present invention can be purified according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies of this invention. See, Buchner, et al., Anal. Biochem. 205:263-270 (1992); Pluckthun, Biotechnology 9:545 (1991); Huse, et al., Science 246:1275 (1989) and Ward, et al., Nature 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well-known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena, et al., Biochemistry 9: 5015-5021 (1970), incorporated by reference herein, and especially as described by Buchner, et al., supra.

Renaturation is typically accomplished by dilution (e.g., 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA

As a modification to the two chain antibody purification protocol, the heavy and light chain regions are separately solubilized and reduced and then combined in the refolding solution. A preferred yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

### Pseudomonas Exotoxin and Other Toxins

Toxins can be employed with antibodies of the present invention to yield chimeric molecules, such as immunotoxins. Exemplary toxins include ricin, abrin, diphtheria toxin and subunits thereof, ribotoxin, ribonuclease, saporin, and calicheamicin, as well as botulinum toxins A through F. These toxins are well known in the art and many are readily available from commercial sources (e.g., Sigma Chemical Company, St. Louis, Mo.). Diphtheria toxin is isolated from Corynebacterium diphtheriae. Ricin is the lectin RCA60 from Ricinus communis (Castor bean). The term also references toxic variants thereof For example, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543 (1972)). The A chain is responsible for inactivating protein synthesis and killing cells. The B chain binds ricin to cell-surface galactose residues and facilitates transport of the A chain into the cytosol (Olsnes, et al., Nature 249:627-631 (1974) and U.S. Pat. No. 3,060,165). Conjugating ribonucleases to targeting molecules for use as immunotoxins is discussed in, e.g., Suzuki et al., Nat Biotech 17:265-70 (1999). Exemplary ribotoxins such as α-sarcin and restrictocin are discussed in, e.g., Rathore et al., Gene 190:31-5 (1997) and Goyal and Batra, Biochem 345 Pt 2:247-54 (2000). Calicheamicins were first isolated from Micromonospora echinospora and are members of the enediyne antitumor antibiotic family that cause double strand breaks in DNA that lead to apoptosis. See, e.g., Lee et al., J. Antibiot 42:1070-87 (1989). The drug is the toxic moiety of an immunotoxin in clinical trials. See, e.g., Gillespie et al., Ann Oncol 11:735-41 (2000).

Ricin is the lectin RCA60 from Ricinus communis (Castor bean). The term also references toxic variants thereof For example, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543 (1972)). The A chain is responsible for inactivating protein synthesis and killing cells. The B chain binds ricin to cell-surface galactose residues and facilitates transport of the A chain into the cytosol (Olsnes, et al., Nature 249:627-631 (1974) and U.S. Pat. No. 3,060,165).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al., Agr. Biol. Chem. 52:1095 (1988); and Olsnes, Methods Enzymol. 50:330-335 (1978)).

Inpreferred embodiments of the present invention, the toxin is Pseudomonas exotoxin (PE). The term “Pseudomonas exotoxin” as used herein refers to a full-length native (naturally occurring) PE or a PE that has been modified. Such modifications may include, but are not limited to, elimation of domain Ia, various amino acid deletions in domains Ib, II and III, single amino acid substitutions and the addition of one or more sequences at the carboxyl terminus such as KDEL (SEQ ID NO:6)and REDL (SEQ ID NO:7). See Siegall, et al., J. Biol. Chem. 264:14256-14261 (1989). In a preferred embodiment, the cytotoxic fragment of PE retains at least 50%, preferably 75%, more preferably at least 90%, and most preferably 95% of the cytotoxicity of native PE. In a particularly preferred embodiment, the cytotoxic fragment is more toxic than native PE.

Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence is provided in commonly assigned U.S. Pat. No. 5,602,095, incorporated herein by reference. The method of action is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall, et al., (1989), supra.

PE employed in the present invention include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE known in the art include PE40, PE38, and PE35.

In preferred embodiments, the PE has been modified to reduce or eliminate non-specific cell binding, frequently by deleting domain Ia as taught in U.S. Pat. No. 4,892,827, although this can also be achieved, for example, by mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”)) exhibits greatly diminished non-specific cytotoxicity. This mutant form of PE is sometimes referred to as PE4E.

PE40 is a truncated derivative of PE as previously described in the art. See, Pai, et al., Proc. Nat'l Acad. Sci. USA 88:3358-62 (1991); and Kondo, et al., J. Biol. Chem. 263:9470-9475 (1988). PE35 is a 35 kD carboxyl-terminal fragment of PE in which amino acid residues 1-279 have deleted and the molecule commences with a met at position 280 followed by amino acids 281-364 and 381-613 of native PE. PE35 and PE40 are disclosed, for example, in U.S. Pat. Nos. 5,602,095 and 4,892,827.

In some preferred embodiments, the cytotoxic fragment PE38 is employed. PE38 is a truncated PE pro-protein composed of amino acids 253-364 and 381-613 which is activated to its cytotoxic form upon processing within a cell (see e.g., U.S. Pat. No. 5,608,039, and Pastan et al., Biochim. Biophys. Acta 1333:C1-C6 (1997)).

While in preferred embodiments, the PE is PE4E, PE40, or PE38, any form of PE in which non-specific cytotoxicity has been eliminated or reduced to levels in which significant toxicity to non-targeted cells does not occur can be used in the immunotoxins of the present invention so long as it remains capable of translocation and EF-2 ribosylation in a targeted cell.

### A. Conservatively Modified Variants of PE

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

### B. Assaying for Cytotoxicity of PE

Pseudomonas exotoxins employed in the invention can be assayed for the desired level of cytotoxicity by assays well known to those of skill in the art. Thus, cytotoxic fragments of PE and conservatively modified variants of such fragments can be readily assayed for cytotoxicity. A large number of candidate PE molecules can be assayed simultaneously for cytotoxicity by methods well known in the art. For example, subgroups of the candidate molecules can be assayed for cytotoxicity. Positively reacting subgroups of the candidate molecules can be continually subdivided and reassayed until the desired cytotoxic fragment(s) is identified. Such methods allow rapid screening of large numbers of cytotoxic fragments or conservative variants of PE.

### Methods of Detecting Cells that Express XAGE-1

In another aspect, this invention provides methods of detecting cells that express XAGE-1. The methods involve detecting either a XAGE-1 transcript or polypeptide. Because cells of many cancers express XAGE-1, methods of detection are useful in the detection of XAGE-1-expressing cancers. In particular, prostate cancer cells, many breast cancer cells, lung cancer cells, ovarian cancer cells, and pancreatic cancer cells can be distinguished from other cells by the expression of XAGE-1, as well as cells from relatively rare sarcomas and muscle cancers.

Tissue samples can be selected from any likely site of primary or metastatic cancer including the prostate or the breast, and distal sites such as the lymph nodes and other organs. Persons of skill in the art are aware that men, as well as women, suffer from breast cancer. Breast cancer in men is relatively rare, representing only about 1% of all breast cancer cases. Because it is uncommon, however, it is frequently diagnosed at a later stage, which affects the chances for survival. Accordingly, improved diagnosis of breast cancer in men is desirable.

In one method, a biopsy is performed on the subject and the collected tissue is tested in vitro. Typically, the cells are disrupted by lysing, sonic disruption, osmotic pressure, freezing and thawing, enzymatic treatment, or other means routine in the art to render the proteins of the nucleus accessible without denaturing them. The cellular contents (or the nuclear contents, if the contents have been fractionated) are then contacted, for example, with an anti-p9 or p16 antibody. Any immune complexes which result indicate the presence of an XAGE-1 protein in the biopsied sample. To facilitate such detection, the antibody can be radiolabeled or coupled to an effector molecule which is radiolabeled. In another method, the cells can be detected in vivo using typical imaging systems. For example, the method can involve the administration to a subject of a labeled composition capable of reaching the cell nucleus. Then, the localization of the label is determined by any of the known methods for detecting the label. Any conventional method for visualizing diagnostic imaging can be used. For example, paramagnetic isotopes can be used for MRI.

### A. Detection of XAGE-1 and xage-1 Proteins

XAGE-1 and xage-1 proteins can be identified by any methods known in the art. In one embodiment, the methods involve detecting a polypeptide with a ligand that specifically recognizes the polypeptide (e.g., an immunoassay). The antibodies of the invention are particularly useful for specific detection of p9 or p16. A variety of antibody-based detection methods are known in the art. These include, for example, radioimmunoassay, sandwich immunoassays (including ELISA), immunofluorescence assays, Western blot, affinity chromatography (affinity ligand bound to a solid phase), and in situ detection with labeled antibodies. Another method for detecting p9 or p16 involves identifying the polypeptide according to its mass through, for example, gel electrophoresis, mass spectrometry or HPLC. Subject samples can be taken from any number of appropriate sources, such as saliva, peritoneal fluid, blood or a blood product (e.g., serum), urine, tissue biopsy (e.g., lymph node tissue), etc.

The p9 or p16 proteins can be detected in cells in vitro, in samples from biopsy and in vivo using imaging systems described above.

### B. Detection of Transcript Encoding XAGE-1

Cells that express XAGE-1 transcript can be detected by contacting the sample with a nucleic acid probe that specifically hybridizes with the transcript, and detecting hybridization. This includes, for example, methods of in situ hybridization, in which a labeled probe is contacted with the sample and hybridization is detected by detecting the attached label. However, the amounts of transcript present in the sample can be small. Therefore, other methods employ amplification, such as RT-PCR. In these methods, probes are selected that function as amplification primers which specifically amplify the XAGE-1 sequences from mRNA. Then, the amplified sequences are detected using typical methods.

The probes are selected to specifically hybridize with XAGE-1 transcripts. Generally, complementary probes are used. However, probes need not be exactly complementary if they have sufficient sequence homology and length to hybridize under stringent conditions.

### Pharmaceutical Compositions

In another aspect, this invention provides pharmaceutical compositions that comprise a pharmaceutically acceptable carrier and a composition of this invention.

In one group of embodiments, the pharmaceutical composition comprises p9 or p16, an immunogenic fragment of one of these proteins, such as a polypeptide comprising a p9 epitope, or a p9 or p16 analog, in an amount effective to elicit a cell-mediated immune response or a humoral response in a subject, e.g., a polypeptide bearing an MHC binding motif. Such pharmaceutical compositions are useful as vaccines in the therapeutic methods of this invention and for preparing antibodies.

In another embodiment, the pharmaceutical composition comprises a nucleic acid molecule comprising a nucleotide sequence encoding p9 or p16 in an amount effective to elicit an immune response against cells expressing p9 or p16 in a subject. Such composition also are useful in the therapeutic methods of this invention.

In yet another embodiment, the pharmaceutical composition may comprise a chimeric molecule comprising a targeting molecule and a detector molecule to detect cells expressing p 16 or p9. If the detector molecule is one capable of binding specifically to a nucleic acid encoding p9 or p16 (such as a DNA binding protein which can bind specifically to DNA encoding p9 or p16), than the composition can be used to detect cells which express that nucleic acid.

The pharmaceutical compositions of this invention can be prepared in unit dosage forms for administration to a subject. The amount and timing of administration are at the discretion of the treating physician to achieve the desired purposes.

In another major group of embodiments, the pharmaceutical compositions of the invention are antibody and/or immunoconjugate compositions of this invention (ie., PE linked to an anti-p9 or p16 antibody). These compositions are particularly suited for parenteral administration, such as intravenous administration or administration into a body cavity or lumen of an organ. For example, ovarian malignancies may be treated by intravenous administration or by localized delivery to the tissue surrounding the tumor. To treat these malignancies, pharmaceutical compositions of this invention comprising anti-p9 or p16 antibodies can be administered directly into the pleural or peritoneal cavities. Anti-p16 antibodies are particularly preferred for use in these compositions.

The compositions for administration will commonly comprise a solution of the antibody and/or immunoconjugate dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Thus, a typical pharmaceutical immunotoxin composition of the present invention for intravenous administration would be about 0.1 to 10 mg per patient per day. Dosages from 0.1 up to about 100 mg per patient per day may be used, particularly if the drug is administered to a secluded site and not into the circulatory or lymph system, such as into a body cavity or into a lumen of an organ Actual methods for preparing administrable compositions will be known or apparent to those skilled in the art and are described in more detail in such publications as REMINGTON'S PHARMACEUTICAL SCIENCE, 19TH ED., Mack Publishing Company, Easton, Pa. (1995).

The compositions of the present invention can be administered to inhibit the growth of cells of XAGE-1 expressing cancers. In these applications, compositions are administered to a patient suffering from a disease, in an amount sufficient to inhibit growth of XAGE-1-expressing cells. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLIOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp.315-339, (1992) both of which are incorporated herein by reference.

Polymers can be used for ion-controlled release of immunoconjugate compositions of the present invention. Various degradable and nondegradable polymeric matrices for use in controlled drug delivery are known in the art (Langer, R., Accounts Chem. Res. 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425-434 (1992); and Pec, et al., J. Parent. Sci. Tech. 44(2):58-65 (1990)). Alternatively, hydroxyapatite has been used as a microcarrier for controlled release of proteins (Ijntema, et al., Int. J. Pharm. 112:215-224 (1994)). In yet another aspect, liposomes are used for controlled release as well as drug targeting of the lipid-capsulated drug (Betageri, et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Among various uses of the immunotoxins of the present invention are included a variety of disease conditions caused by specific human cells that may be eliminated by the toxic action of the fusion protein. One preferred application for the immunotoxins of the invention is the treatment of malignant cells expressing XAGE-1. Exemplary malignant cells include ovarian, lung, prostate, breast, and pancreatic cancers, as well as XAGE-1-expressing muscle and bone cancers.

### Diagnostic Kits and In Vitro Uses

In another embodiment, this invention provides for kits for the detection of p9 or p16 or an immunoreactive fragment thereof, (i.e., collectively, a “xage-1 protein”) in a biological sample. A “biological sample” as used herein is a sample of biological tissue or fluid that contains an xage-1 protein. Such samples include, but are not limited to, tissue from biopsy, sputum, blood, and blood cells (e.g., white cells). Biological samples also include sections of tissues, such as frozen sections taken for histological purposes.

Kits will typically comprise an anti-p9 or p16 antibody of the present invention. In some embodiments, the anti-p9 or p16 antibody may be an anti-p9 or p16 Fv fragment, such as a scFv fragment or a dsFv.

In addition the kits will typically include instructional materials disclosing means of use of an antibody of the present invention (e.g. for detection of prostate cancer cells in a sample). The kits may also include additional components to facilitate the particular application for which the kit is designed. Thus, for example, the kit may additionally contain means of detecting the label (e.g. enzyme substrates for enzymatic labels, filter sets to detect fluorescent labels, appropriate secondary labels such as a sheep anti-mouse-HRP, or the like). The kits may additionally include buffers and other reagents routinely used for the practice of a particular method. Such kits and appropriate contents are well known to those of skill in the art.

In one embodiment of the present invention, the diagnostic kit comprises an immunoassay. As described above, although the details of the immunoassays of the present invention may vary with the particular format employed, the method of detecting p9 or p16 in a biological sample generally comprises the steps of contacting the biological sample with an antibody which specifically reacts, under immunologically reactive conditions, to p9 or to p16. Since p9 is an intracellular protein, cells to be tested for p9 will typically be disrupted prior to contact with the antibody. Conveniently, disruption can be by sonication, although other methods known in the art may also be used so long as they do not denature p9 or interfere with antibody binding. The antibody is allowed to bind to p9 or to p16 under immunologically reactive conditions, and the presence of the bound antibody is detected directly or indirectly.

The antibodies provided herein will be especially useful as diagnostic agents and in in vitro assays to detect the presence of p9 or p16 in biological samples. For example, the antibodies made by the methods taught herein can be used as the targeting moieties of immunoconjugates in immunohistochemical assays to determine whether a sample contains cells expressing p9 or p 16. If the sample is one taken from a tissue of a patient which should not normally express p9 or p16, detection of one of those proteins would indicate, for example, that the patient has a cancer characterized by the presence of XAGE-1-expressing cells, in a patient not previously known to have such a cancer or, for a patient under treatment for such a cancer, that the treatment has not yet been successful at eradicating it. In preferred embodiments, the cancer is not a bone or muscle cancer.

In some embodiments, the biological sample is from an adult. Since the bone and muscle cancers in which XAGE-1 is expressed are most frequently found in children, the detection of cells expressing XAGE-1 in a sample biopsied from an adult is very unlikely to indicate the presence of a bone or muscle cancer and much more likely to indicate the presence of a lung, prostate, or breast cancer. As noted earlier, XAGE-1 is expressed in normal tissues in high amounts only in the testes; it is found in very low levels in the lung and in peripheral blood cells. Thus, detection of very low levels of XAGE-1 in a sample biopsied from the lung or bone marrow would not necessarily indicate the presence of an XAGE-1 expressing cancer, but high levels would.

In another set of uses for the invention, immunotoxins targeted by antibodies of the invention can be used to purge targeted cells from a population of cells in a culture. Thus, for example, cells cultured from a patient having a XAGE-1-expressing cancer can be purged of cancer cells by contacting the culture with immunotoxins which target cells expressing p9 or p16.

### EXAMPLES

### Example 1

### Materials and Methods

Tissues and cell lines: Ewings tumor tissue was obtained from frozen specimens obtained from patients treated at the National Cancer Institute. Rhabdomyosarcoma tumor tissue was obtained from the Cooperative Human Tissue Network, CCG. All alveolar rhabdomyosarcoma tumor specimens were found to express the PAX-3-FKHR fusion transcript by RT-PCR, Osteosarcoma cell lines were obtained from the American Type Culture Collection. Ewing sarcoma cell lines RD-ES, TC-32, TC-71 and 5838 have been previously described, and all contain EWS-FLI-1 fusion transcripts (Van Valen, F. Ewing's Sarcoma Family of Tumors in Human Cell Culture, Vol. I, Boston MA (Kluwer Academic Publishers 1999). LD, LG, JM, and SB are cell lines established in our laboratories directly from tumor specimens. The cell line JM does not express an EWS-ETS fusion transcript.

Northern Blots and RNA dot blot: RNA was extracted either from tumor tissue using Trizol (Life Technologies) or from cell lines using RNAeasy from Qiagen. 20 μg of total RNA was used for northern blot analysis of sarcoma tumors. The multiple tissue mRNA dot blot and the normal tissue northern blot were purchased from Clontech. The 450 bp probe used for hybridization was generated from EST clone af89d01.s1 by digestion with EcoRI and NotI. The hybridization was conducted as follows: the RNA containing membranes were blocked for 3 hours at 45° C. in hybridization solution. Probes labeled with ³²P either by random primer extension or by end labeling (Lofstrand Labs Limited) were added to the membrane and hybridized for 15 hour at 45° C. Membranes were washed twice with 2×SSC/0.1% SDS at room temperature and twice with 0.1×SSC/0.1% SDS at 65° C. The membranes were exposed to X-ray film for 1-2 days before development.

The Southern blot of human chromosomes (Oncor, Gaithersburg, Md.) was conducted using same probe and same hybridization conditions as for Northern blot.

RT-PCR was performed on cDNA from 24 different human tissues using human rapid-scan gene expression panels (Origene Inc., Rockville, Md.). The thermocycling protocol was: initial denaturation at 94° C. for 3 minutes; 35 cycles of denaturation at 94° C. for 1 minute, annealing at 65° C. for 1 minute, and elongation at 72° C. for 3 minutes. The PCR reactions were analyzed on agarose gels and specific products were cloned into TA vectors (Invitrogen) and sequenced on an automated capillary sequencer, using Perkin-Elmer's drhodamine termintator cycle sequencing kit (Perkin-Elmer Applied System).

The primers used were:

xa-1:5′-CAGCTTGTCTTCATTTAAACTTGTGGTTGC-3′ (SEQ ID NO:8);

xa-2:5′-TCCCAGGAGCCCAGTAATGGAGA-3′ (SEQ ID NO:9);

xa-8:5′-ACCTGGGAAGGAGCATAGGA-3′ (SEQ ID NO:10); and xa-10:5′-CTTTATTGAGATAGTTTAAGTCAAATATCTAA-3′ (SEQ ID NO:11). The oligo-nucleotides were synthesized by Sigma-Genosys.

### Example 2

### Expression of XAGE-1 in Normal Tissues

To determine the relative expression of XAGE-1 mRNA in different tissues and tumors, a mRNA dot blot (Clontech, Palo Alto, Calif.) analysis was conducted using a full insert of EST 89d01.s1 as a labeling probe. Among the 61 different samples of normal tissues and 7 fetal tissues including lung, brain, liver, heart and spleen, the expression of XAGE-1 was only detected in testis. This result indicates that XAGE-1, like other cancer-testis antigens, is present in testis.

To verify the specificity of XAGE-1 expression, RT-PCR analysis was conducted by using the human rapid-scan panel with primers xa-1 and xa-2. A 275 bp fragment in testis was detected among the 24 different tissues analyzed. Unexpectedly, the 275 bp fragment was also present at lower amounts in normal lung and peripheral leukocytes (PBL). Extremely weak expression of XAGE-1 was detected in bone marrow, spleen and skin. To compare the relative level of XAGE-1 in testis, lung and PBLs, different dilutions of cDNA were analyzed in the same rapid-scan panel. The mRNA present in testis was about 10-100 times higher than in lung, and more than 100 times higher than in peripheral leukocytes.

Since XAGE-1 is highly abundant in testis, and expressed at a low level in lung and PBLs, we attempted to determine the transcript size in these different tissues. Northern blot analysis was conducted by using the same probe as that used for the RNA dot blot. A single band of 700 bp was revealed in the testis. However, no signal was detected in lung and peripheral leukocytes. This result is probably due to the low level of XAGE-1 expression in lung and peripheral leukocytes, because the Northern blot analysis is much less sensitive than RT-PCR to detect the expression of XAGE-1. These results are consistent with the RNA dot blot analysis as described above.

### Example 3

### XAGE-1 Expression in Ewing's Sarcoma, Rhabdomyosarcoma and Osteosarcoma

Analysis of the EST database predicts that XAGE-1 is present in Ewing's sarcoma and alveolar rhabdomyosarcoma To confirm the database prediction experimentally, we first determined whether XAGE-1 was present in the various Ewing's cell lines by Northern blot analysis. A single band of 700 bp was detected in the 7/8 cell lines. XAGE-1 was not expressed in cell line JM, which is a mouse xenograft tumor derived from a Ewing's sarcoma which has lost the chromosome translocation t (Lucas, S. et al., Cancer Res., 58:743-752 (1998); Watari, K. et al., FEBS Lett., 466: 367-371 (2000)). Cell line 5838 had an extra band with a size of 1.2 kb. This band might be due to alternate splicing or use of an alternate polyadenalytion signal in XAGE-1 gene. XAGE-1 was present in 2/5 osteosarcoma cell lines with the SAOS cell line showing relatively low expression.

To address whether XAGE-1 was present in human patient samples, a Northern blot hybridization analysis was conducted. Out of 9 patients with Ewing's sarcoma, 4 of them (patients 5,6,7 and 8) expressed XAGE-1 with a single 700 bp band. Patients 1, 5, 6, 7, 8 and 9 expressed EWS-FLI-1 transcript, an indication of chromosome translocation (Sorensen, P. H. et al., Nature Genetics., 6:146-151 (1991)). XAGE-1 was not expressed in all of the patient samples with the chromosome translocation. However, samples that did not express either an EWS-FLI-1 or an EWS-ERG fusion transcript also did not express XAGE-1. XAGE-1 was also expressed in 1/1 patient samples with alveolar rhabdomyosarcoma and patient samples of 1/3 embryonal rhabdomyosarcomas but not in the normal controls. These data together indicates that XAGE-1 is expressed in nearly half of the sarcoma patient samples.

### Example 4

### Chromosome Localization of XAGE-1

Most of the CT antigens are localized on the X chromosome with the exception of SCP-1 which is located on chromosome 1 (De Smet, C. et al., Eye., 11:243-248 (1997); Tureci, O. et al., Proc. Natl. Acad. Sci. USA., 95:5211-5216 (1998)). To find where XAGE-1 is localized, Southern blot hybridization was performed on a human chromosome blot using the same probe as that for dot blot and Northern blot. One strong band was detected on the X chromosome, and there were no other cross hybridizing bands found on the blot. This result indicates that the XAGE-1 gene is located on the X chromosome and that there is not a very strong homology with the other predicted XAGE members, XAGE- 2 and XAGE-3, because under stringent hybridization conditions, XAGE-2 and XAGE-3 were not detected.

### Example 5

### RACE-PCR Determination of Full-Length cDNA of XAGE-1 and Peptide Sequences

To obtain the full-length XAGE-1 cDNA sequence, RACE-PCR was performed using primers localized in the EST contig and total RNA from Ewing's cell line TC71. The longest RACE product contains an additional 184 nucleotides at the 5′ end compared to the EST contig sequence. The correct cDNA sequence was confirmed by sequencing the PCR product from primers xa8 and xa10. The XAGE-1 cDNA is 611 bp in length excluding the poly (A) tail and contains 438 nucleotides in the coding region, flanked by 85 bp in the 5′ untranslated region and 88 bp in the 3′ untranslated region.

The longest ORF indicates that the encoded xage-1 protein consists of 146 amino acids residues with a molecular weight of 16.3 kD. This protein has been termed “p16” herein. Hydrophilicity analysis of the p16 amino acid sequence indicates a hydrophobic sequence in the N-terminal end, suggesting the protein is membrane-associated. Analysis of the protein sequence reveals no possible post-translational modifications by searching GCG Lite. This protein did not show overall sequence homology with any peptide recorded in the data banks. However, alignment of the amino acid sequence of XAGE-1 p16 with PAGE4 (Brinkmann, U. et al., Proc. Natl. Acad. Sci. USA., 95:10757-10762 (1998)) and PAGE1 (Chen, M. E. et al., J. Biol. Chem., 273:17618-17625 (1998)) (renamed GAGE9 ) (Backer, O. et al., Cancer Res., 59:3157-3165 (1991)) reveals a striking homology in the C terminal end of these proteins, suggesting that XAGE-1 encodes a distinct protein which could share structural or functional features with other GAGE/PAGE family members.

### Example 6

### Primer Extension Analysis Reveals Two Start Sites for the XAGE-1 Transcript

To further verify the transcription initiation start site of XAGE-1, a primer extension analysis was performed using total RNA isolated from TC71, a Ewing's sarcoma cell line, and normal testis; both of which were previously shown to express XAGE-1. The primer Xagext.3 is located in the first exon of XAGE-1 to ensure that the primer extension product proper aligns with the DNA sequencing ladder, which was cloned from genomic DNA. Surprisingly, the primer extension product derived from the Xagext.3 primer corresponds to a transcription initiation start site located 58 bp downstream of the first ATG translational start codon (FIG. 1). To map the 5′ most transcriptional start site of XAGE-1, the primer Xagext.4 was used. The most abundant primer extension product derived from the Xagext.4 primer corresponds to a guanine located 11 bp upstream from the start of the longest RACE-PCR product reported in the previous Example. This primer extension analysis reveals that there are two distinct starts sites for the XAGE-i transcript

### Example 7

### Antibodies to XAGE-1 Proteins and Demonstration that XAGE-1 p9 is Expressed

Polyclonal antibodies were generated against a Pseudomonas exotoxin (ΔPE)-XAGE fusion protein according to the procedure described by Bruggemann et al. BioTechniques 10:202-209 (1991). Briefly, a ΔPE-XAGE fusion protein was made by cloning a 3′ XAGE-1 fragment encoding the 109 C-terminal residues of XAGE-1 in frame with the 3′ end of a mutant ΔPE gene containing a single codon deletion that renders the encoded enzyme catalytically inactive. The ΔPE-XAGE protein was overexpressed in E. coli BL21(λDE3), and inclusion bodies containing the fusion protein were isolated and washed. Female white New Zealand rabbits were immunized with the purified inclusion bodies. The antiserum from the rabbits was purified by running it over a protein A column. Captured antibodies were then run over an immobilized E. coli lysate column, according to the manufacturer's instructions (Pierce).

A DNA fragment including the XAGE-1 open reading frame and 21 base pairs 5′ of the first putative ATG start codon was amplified by PCR and cloned into the HindIII and XhoI sites of pcDNA3 (Invitrogen) to allow expression from the CMV promoter. The resulting plasmid was designated as pCMV-XAGE. Human embryonic kidney cells, 293T, were transfected with either pCMV-XAGE or pcDNA3 by the protocol according to Pear et al., Proc. Natl. Acad. Sci. USA 90, 8392-8396 (1993). Briefly, 293T cells were transfected by using CaPO₄ precipitation. The cells were harvested 48 hours post-transfection, and whole cell protein extracts were prepared from cells containing vector only, pCMV-XAGE, or untransfected cells. A Westem-immunoblot analysis was performed on the protein extracts. Whole cell protein extracts (40 μg) were run on a 16.5% polyacrylamide gel (Bio-Rad) and transferred to a polyvinylidene fluoride (PVDF) membrane (Millipore). The membranes were probed with 10 μg/ml of either serum taken from animals prior to exposure to APE-XAGE or antiserum from animals injected with APE-XAGE. A chemiluminescence Western blotting kit was used to detect XAGE on the membrane according to the manufacturer's instructions (Roche Molecular Biochemicals).

The results revealed a 9 kDa band in the pCMV-XAGE sample which was not present in the extracts prepared from untransfected cells or those transfected only with vector. The size of the protein demonstrates that translation of the XAGE-1 transcript in 293T cells begins with the second ATG in the reading frame corresponding to residue 66 of the full length sequence set forth in FIG. 1.

### Example 8

### Expression of XAGE-1 in Cancers Other than Ewing's Sarcoma, Rhabdomyosarcoma and Osteosarcoma

A series of studies were conducted to determine the expression of XAGE-1 in important human cancers. Multiple cell lines, tissues, and patient samples were assayed for the expression of XAGE-1 by the methods discussed in the preceding Examples. The results are set forth in the Tables, below. RT-PCR means reverse transcriptase polymerase chain reaction.

XAGE-1 expression was examined by RT-PCR analysis using PCR primers to the XAGE-1 gene. To compare relative levels of XAGE-1 expression between cell lines, separate PCR reactions were performed using primers to β-actin to verify the quality of the generated cDNA. XAGE-1 was expressed in two of three prostate cell lines, LNCaP and DU145, but not in PC3. Expression of XAGE-1 was not detected in any of the assayed estrogen receptor positive (“ER+”) breast cancer cell lines, CRL1500, MCF7, and HTB-20 (Table 1). However, the ER-breast cancer cell lines, MDA-MB-231, HTB-20, and MDA-MB-268, all expressed XAGE-1. (It should be noted that it has not been determined whether ER+ breast cancers other than the cell lines studied express XAGE-1.) Certain tumor types rarely express CT-antigens, such as gastrointestinal carcinomas, colorectal carcinomas, renal cancers, leukemias and lymphomas (De Smet, C. et al., Eye, 11: 243-248 (1997); Van den Eynde, B. J. et al., Curr Opin Immunol., 9: 684-93 (1997); Chen, Y. T. et al., Cancer J. Sci Am., 5: 16-7 (1999)). Similarly, XAGE-1 is not expressed in any of the studied colon, rectum, colorectal, or Burkitt's lymphoma cancer cell lines. Surprisingly, however, XAGE-1 was expressed in a T cell lymphoma cell line, HUT102 and U937, a histiocytic lymphoma cell line. Expression in lymphomas is rare for CT antigens.

The results from a cancer profiling array indicated that XAGE-1 is expressed in lung squamous cell carcinomas and lung adenocarcinomas. To corroborate these results, total RNA was isolated from frozen patient tumor samples and subjected to RT-PCR analysis using primers to XAGE-1 (Table 5). XAGE-1 was expressed in two of three lung squamous cell carcinomas and two of three lung adenocarcinomas. Other CT antigen genes, such as MAGE, BAGE, and GAGE, are expressed in a significant proportion of non-small cell lung carcinomas (NSCLC) (De Smet, C. et al., Eye, 11: 243-248 (1997); Van den Eynde, B. J. et al., Curr Opin Immunol., 9: 684-93 (1997)). In addition, NY-ESO-1 is expressed in both NSCLC and small cell lung cancers (SCLC) (Lee, L. et al., Cancer J. Sci Am., 5: 20-5 (1999)). To address whether XAGE-1 is also expressed in NSCLC and SCLC, total RNA was isolated from frozen tumor samples, and XAGE-1 expression was determined by RT-PCR analysis using primers to XAGE-1 (Table 5). XAGE-1 was expressed in all three of the SCLC samples analyzed and in both of the NSCLC samples. Similar to other CT antigens, XAGE-1 is expressed in NSCLC and SCLC.

### Example 9

### In Situ Hybridization

### Materials and Methods for In Situ Hybridization

Slide preparation: The paraffin embedded breast and prostate tissue sections were deparaffinized by placing the slides over a slide warmer at 65* C for 1 hr. The slides were then placed in two changes of Xylene for 5 min each and air-dried. They were then rinsed in two changes of absolute alcohol for 5 min each and air-dried.

Probe preparation: A 592 bp XAGE-1 DNA fragment including 83 bp upstream of the first ATG translational start codon to the first polyA signal sequence (see FIG. 1A) was cloned into the plasmid pBluescript and biotinylated. Biotinylated pBluescript without any insert was used as a negative control. Probes were labeled using the BioNick Labeling System (Life Technologies- Cat. No.18247-015) following the vendors recommendation with a few minor modifications. The probes were incubated at 16* C for 3 hr and not for 1 hr as suggested by the vendor. The unincorporated nucleotides from the labeled DNA probes were removed by ethanol precipitating the probes three times. The prepared probes are very stable and can be stored at −20* C.

Hybridization: Slides were hybridized using the in situ Hybridization and Detection System (Life Technologies—Cat.No.18250-019) following the vendor's recommendation with a few modification (Kumar V., Collins F. H., Insect Mol Biol.; 3(1):41-7 (1994)). The slides were counter stained using 0.2% Light Green stain, rinsed through a series of alcohol grades and mounted in Cytoseal. They were photographed at a 10× magnification with a digital camera mounted on a Nikon Eclipse E800 Microscope.

### Results of In Situ Hybridizations

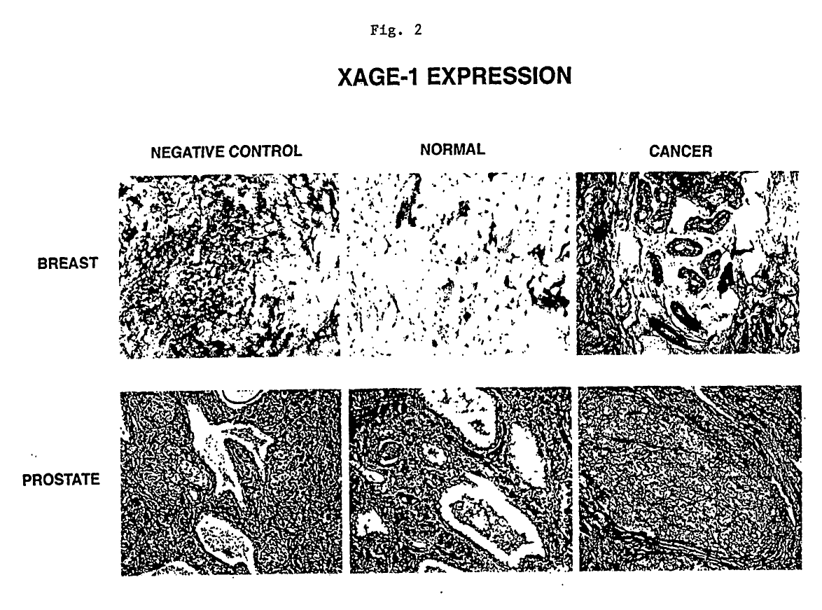

In situ hybridization using XAGE-1 as a probe was performed on normal breast and breast tumor tissue sections, as well as tissue sections of normal and prostate cancer. As a negative control, pBlueScript containing no insert was used as a probe for the breast and prostate tissue sections, and no signal was detected. The normal breast section showed weak expression of XAGE-1 while the signal in the breast tumor was very intense (FIG. 2, top row, compare middle and right hand photos). The normal prostate section showed a very weak signal in the epithelial cells, yet the prostate tumor showed moderately intense signal for XAGE-1 FIG. 2, bottom row, compare middle and right hand photos).

TABLE 1
Expression of XAGE-1 in human cancer cell lines.

| | | Level of XAGE-1 |
|---|---|---|
| Cell line | Cancer type | Expression |
| LNCaP | Prostate | ++++ |
| PC3 | Prostate | − |
| DU145 | Prostate | ++++ |
| CRL1500 | Breast (ER+) | − |
| MCF7 | Breast (ER+) | − |
| HTB-20 | Breast (ER+) | − |
| MDA-MB-231 | Breast (ER−) | ++++ |
| HTB-30 | Breast (ER−) | ++ |
| MDA-MB-468 | Breast (ER−) | + |
| OVCAR | Ovarian | +++ |
| FEM-X | Melanoma | + |
| HUT102 | T cell lymphoma | + |
| U937 | Histiocytic | +++ |
| | Lymphoma | |
| Daudi | Burkitt's lymphoma | − |
| JD38 | Burkitt's lymphoma | − |
| Raji | Burkitt's lymphoma | − |
| A-172 | Glioblastoma | + |
| IMR-32 | Neuroblastoma | − |
| Colo 205 | Colon | − |
| LOVO | Colon | − |
| SW403 | Rectum | − |
| SW480 | Colorectal adeno | − |
| SW620 | Colorectal adeno | − |

Either total or polyA RNA was isolated from the tumor cell lines. Expression levels were determined by RT-PCR using Xa-1 and Xa-2 primers to the XAGE-1 gene. Separate PCR reactions were performed using actin primers to verity the quality of the generated cDNA. Relative levels of expression are indicated by the number of +'s. Minus (−) indicates no expression. “ER” stands for “estrogen receptor,” “ER+” indicates that the cells were positive for the estrogen receptor, “ER−” indicates that the cells were negative for the receptor.

TABLE 2
XAGE Expression in Xenografts
(human tumors cells introduced into mice)

| | |
|---|---|
| Lung carcinoma | (+) |
| Lung carcinoma | ++++ |
| Colon adenocarcinoma | + |
| Colon adenocarcinoma | + |
| Prostate adenocarcinoma | + |
| Breast carcinoma | ++ |
| Ovarian carcinoma | (+) |
| Pancreatic adenocarcinoma | ++ |

TABLE 3
XAGE-1 Expression in a Panel of Normal Breast and Breast
Cancer Samples From Patients, Tested by RT-PCR

| Normal breast | (0/12) positive |
|---|---|
| Breast tumor | −(5/12), +(3/12), ++++(4/12) |

TABLE 4
XAGE-1 Expression Tested by by Dot Blot

| | |
|---|---|
| Positive: | Leukemia K-562 |
| | Lung carcinoma A549 |
| Negative: | Leukemia HL-60, MOLT-4 |

TABLE 5
Expression of XAGE-1 in lung cancers.

| | | No. of cancers | % of samples |
|---|---|---|---|
| | Total no. | which | expressing |
| Lung Cancer Type | tested | express XAGE-1 | XAGE-1 |
| Small cell carcinoma | 3 | 3 | 100 |
| Non-small cell carcinoma | 2 | 2 | 100 |
| Squamous cell carcinoma | 3 | 2 | 67 |
| Adenocarcinoma | 3 | 2 | 67 |

Total RNA was isolated from frozen tumor samples, and expression levels were determined by RT-PCR using primers Xa-1 and Xa-2 to the XAGE-1 gene. Separate PCR reactions were performed using actin primers to confirm the quality of the generated cDNA.

While specific examples have been provided, the above description is illustrative and not restrictive. Many variations of the invention will become apparent to those skilled in the art upon review of this specification. The scope of the invention should, therefore, be determined not with reference to the above description, but instead should be determined with reference to the appended claims along with their full scope of equivalents.

All publications and patent documents cited herein are incorporated by reference in their entirety for all purposes to the same extent as if each individual publication or patent document were so individually denoted. Citation of various references in this document is not an admission that any particular reference is considered to be “prior art” to the invention.

What is claimed is:

1. An isolated polypeptide comprising an amino acid sequence selected from the group consisting of a xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9.

2. An isolated polypeptide of claim 1, wherein the polypeptide comprises the sequence of p9.

3. An isolated polypeptide of claim 1, wherein the polypeptide comprises the sequence of an immunogenic fragment of p9.

4. An isolated polypeptide of claim 1, which polypeptide has at least 90% sequence identity to p9 and is specifically recognized by an antibody which specifically recognizes p9.

5. An isolated polypeptide of claim 1, which polypeptide has at least 90% sequence identity with xage-1 p9 and which, when processed and presented in the context of Major Histocompatibility Complex molecules, activates T lymphocytes against cells which express xage-1 p9.

6. A composition comprising a polypeptide of claim 1 and a pharmaceutically acceptable carrier.

7. A composition comprising a polypeptide of claim 2 and a pharmaceutically acceptable carrier.

8. A composition comprising a polypeptide of claim 3 and a pharmaceutically acceptable carrier.

9. A composition comprising a polypeptide of claim 4 and a pharmaceutically acceptable carrier.

10. A composition comprising a polypeptide of claim 5 and a pharmaceutically acceptable carrier.

11. An isolated, recombinant nucleic acid molecule comprising a nucleotide sequence encoding a polypeptide having the amino acid sequence of an xage-1 p9 protein (“p9,” SEQ ID NO:2), an immunogenic fragment thereof a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9.

12. The isolated, recombinant nucleic acid molecule of claim 11, which encodes a polypeptide comprising the sequence of xage-1 p9.

13. The isolated, recombinant nucleic acid molecule of claim 11, wherein the polypeptide is an immunogenic fragment of xage-1 p9.

14. The isolated, recombinant nucleic acid molecule of claim 11, wherein the polypeptide has at least 90% sequence identity to xage-1 p9 and which is specifically recognized by an antibody which specifically recognizes xage-1 p9.

15. The isolated recombinant nucleic acid molecule of claim 11, wherein the polypeptide has at least 90% sequence identity with xage-1 p9 and which, when processed and presented in the context of Major Histocompatibility Complex molecules, activates T lymphocytes against cells which express xage-1 p9.

16. A host cell comprising an expression vector comprising a promoter operatively linked to a nucleotide sequence encoding a polypeptide selected from the group consisting of: xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9.

17. A use of an isolated polypeptide comprising an amino acid sequence selected from the group consisting of a xage-1 p9 protein (“p9” (SEQ ID NO:2)), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9, for the manufacture of a medicament for activating T lymphocytes against cells expressing xage-1 p9.

18. A use of claim 17, wherein said isolated polypeptide comprises the sequence of p9.

19. A use of claim 17, wherein the polypeptide comprises the sequence of an immunogenic fragment of p9.

20. A use of claim 17, wherein the polypeptide has at least 90% sequence identity to p9 and is specifically recognized by an antibody which specifically recognizes p9.

21. A use of claim 17, wherein the polypeptide has at least 90% sequence identity with xage-1 p9 and which, when processed and presented in the context of Major Histocompatibility Complex molecules, activates T lymphocytes against cells which express xage-1 p9.

22. A use of an isolated, recombinant nucleic acid molecule comprising a nucleotide sequence encoding a polypeptide selected from the group consisting of a polypeptide having the amino acid sequence of an xage-1 p9 protein (“p9,” SEQ ID NO:2), an immunogenic fragment thereof, a polypeptide with at least 90% sequence identity to p9 and which is specifically recognized by an antibody which specifically recognizes p9, and a polypeptide which has at least 90% sequence identity with p9 and which, when processed and presented in the context of Major Histocompatibility Complex molecules, activates T lymphocytes against cells which express p9, for the manufacture of a medicament for activating T lymphocytes against cells expressing xage-1 p9.

23. A use of claim 22, wherein the cells expressing xage-1 p9 are cells of cancers other than Ewing's sarcoma or alveolar rhabdomyosarcoma

24. A use of claim 22, wherein the cells expressing xage-1 p9 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells.

25. A use of claim 22, wherein the isolated, recombinant nucleic acid molecule encodes the sequence of xage-1 p9 (SEQ ID NO:2).

26. A use of claim 22, wherein the isolated, recombinant nucleic acid molecule encodes an immunogenic fragment of xage-1 p9.

27. A use of claim 22, wherein isolated, recombinant nucleic acid molecule encodes a polypeptide with at least 90% sequence identity to xage-1 p9 (SEQ ID NO:2) and which is specifically recognized by an antibody which specifically recognizes xage-1 p9.

28. A use of claim 22, wherein the isolated recombinant nucleic acid molecule encodes a polypeptide with at least 90% sequence identity to xage-1 p9 (SEQ ID NO:2) and which, when processed and presented in the context of Major Histocompatibility Complex molecules, activates T lymphocytes against cells which express xage-1 p9.

29. A method of activating T lymphocytes against cells expressing xage-1 p9 (SEQ ID NO:2), the method comprising administering to a subject a composition, which composition is selected from the group consisting of: an isolated polypeptide having the amino acid sequence of xage-1 p9, an immunogenic fragment thereof, a polypeptide with at least 90% sequence identity to xage-1 p9 and which is specifically recognized by an antibody which specifically recognizes xage-1 p9, a polypeptide which has at least 90% sequence identity with xage-1 p9 and which, when processed and presented in the context of Major Histocompatibility Complex molecules, activates T lymphocytes against cells which express xage-1 p9, an isolated nucleic acid encoding one of these polypeptides, an antigen presenting cell pulsed with a polypeptide comprising an epitope of xage-1 p9, an antigen presenting cell sensitized in vitro to xage-1 p9, an antigen presenting cell sensitized in vitro to an immunogenic fragment of xage-1 p9, an antigen presenting cell sensitized in vitro to a polypeptide with at least 90% sequence identity to xage-1 p9 which is specifically recognized by an antibody which specifically recognizes xage-1 p9, and an antigen presenting cell sensitized in vitro to polypeptide which has at least 90% sequence identity with xage-1 p9 which, when processed and presented in the context of Major Histocompatibility Complex molecules, activates T lymphocytes against cells which express xage-1 p9.

30. A method of claim 29 comprising administering to the subject xage-1 p9 or an immunogenic fragment thereof.

31. A method of claim 29 wherein the polypeptide has at least 90% sequence identity to xage-1 p9 and is specifically recognized by an antibody which specifically recognizes xage-1 p9.

32. A method of claim 29, wherein the polypeptide has at least 90% sequence identity with xage-1 p9 and, when processed and presented by an antigen presenting cell in conjunction with an MHC molecule, activates T lymphocytes against cells expressing xage-1 p9.

33. The method of claim 29 wherein the composition is administered to a subject who suffers from a cancer selected from prostate cancer cells, lung cancer cells, ovarian cancer cells, breast cancer cells, glioblastoma cells, pancreatic cancer cells, T cell lymphoma cells, melanoma cells, and histocytic lymphoma cells.

34. The method of claim 33, wherein the composition is administered to a subject suffering from a lung cancer selected from the group consisting of small cell carcinoma, non-small cell carcinoma, squamous cell carcinoma, and adenocarcinoma.

35. The method of claim 33, wherein the composition is administered to a subject suffering from a cancer selected from the group consisting of Ewing's sarcoma, rhabdomyosarcoma and osteosarcoma.

36. The method of claim 29 wherein the administration comprises sensitizing CD8+ cells in vitro to an epitope of an xage-1 p9 protein (SEQ ID NO:2) and administering the sensitized cells to the subject.

37. The method of claim 29, further comprising co-administering to the subject an immune adjuvant selected from non-specific immune adjuvants, subcellular microbial products and fractions, haptens, immunogenic proteins, immunomodulators, interferons, thymic hormones and colony stimulating factors.

38. The method of claim 29, further comprising administering an antigen presenting cell pulsed with a polypeptide comprising an epitope of xage-1 p9 (SEQ ID NO:2).

39. The method of claim 29 comprising administering a nucleic acid sequence encoding polypeptide comprising an epitope of xage-1 p9 (SEQ ID NO:2), which nucleic acid is in a recombinant virus.

40. The method of claim 29, comprising administering a nucleic acid sequence encoding a polypeptide comprising an epitope of an xage-1 p9 protein (SEQ ID NO:2).

41. The method of claim 29, comprising immunizing the subject with a expression vector that expresses a polypeptide comprising an epitope of an xage-1 p9 protein (SEQ ID NO:2), which expression vector is in an autologous recombinant cell.

42. The method of claim 29, wherein the CD8+ cells are T_{C} cells.

43. The method of claim 29 wherein the T_{C} cells are tumor infiltrating lymphocytes.

44. A method for determining whether a subject has an xage-1 p9 expressing cancer, comprising taking a cell sample from said subject from a site other than the testes, and determining whether a cell in said sample contains a nucleic acid transcript encoding xage-1 p9 (SEQ ID NO:2), or detecting xage-1 p9 produced by translation of the transcript, whereby detection of the transcript or of the protein in said sample indicates that the subject has an xage-1 p9 expressing cancer.

45. The method of claim 44, comprising detecting the transcript.

46. The method of claim 44, comprising detecting the protein.

47. The method of claim 44, comprising contacting RNA from the cell with a nucleic acid probe that specifically hybridizes to the transcript under hybridization conditions, and detecting hybridization.

48. The method of claim 44, comprising disrupting said cell and contacting a portion of the cell contents with a chimeric molecule comprising a targeting moiety and a detectable label, wherein the targeting moiety specifically binds to xage-1 p9 (SEQ ID NO:2), and detecting the label bound to the xage-1 p9.

49. The method of claim 44, wherein the cell is taken from a lymph node.

50. An isolated polypeptide comprising an amino acid sequence selected from the group consisting of a xage-1 p16 protein (“p16,” SEQ ID NO:4), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16.

51. An isolated polypeptide of claim 50, wherein the polypeptide comprises the sequence of p16.

52. An isolated polypeptide of claim 50, wherein the polypeptide comprises the sequence of an immunogenic fragment of p16.

53. An isolated polypeptide of claim 50, which polypeptide has at least 90% sequence identity to p16 and is specifically recognized by an antibody which specifically recognizes p16.

54. An isolated polypeptide of claim 50, which polypeptide has at least 90% sequence identity with p16 and which, when processed and presented in the context of Major Histocompatibility Complex molecules, activates T lymphocytes against cells which express p16.

55. A composition comprising a polypeptide of claim 50 and a pharmaceutically acceptable carrier.

56. An isolated, recombinant nucleic acid molecule comprising a nucleotide sequence encoding a polypeptide selected from the group consisting of a polypeptide having the amino acid sequence of an xage-1 p16 protein (“p16”, (SEQ ID NO:4)), an immunogenic fragment thereof a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16.

57. A isolated, recombinant nucleic acid molecule of claim 56, which molecule encodes a polypeptide having the sequence of xage-1 p16.

58. A isolated, recombinant nucleic acid molecule of claim 56, which molecule encodes a polypeptide which is an immunogenic fragment of xage-1 p16.

59. A isolated, recombinant nucleic acid molecule of claim 56, wherein the polypeptide has at least 90% sequence identity to xage-1 p16 and which is specifically recognized by an antibody which specifically recognizes xage-1 p16.

60. An expression vector, said vector comprising an isolated, recombinant nucleic acid molecule of claim 56 operatively linked to a promoter.

61. A use of an isolated polypeptide comprising an amino acid sequence selected from the group consisting of a xage-1 p16 protein (“p16” (SEQ D NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16, for the manufacture of a medicament for activating T lymphocytes against expressing xage-1 p16.

62. A use of claim 61, wherein said isolated polypeptide comprises the sequence of p16.

63. A use of claim 61, wherein the polypeptide comprises the sequence of an immunogenic fragment of p16.

64. A use of claim 61, wherein the polypeptide has at least 90% sequence identity to p16 and is specifically recognized by an antibody which specifically recognizes p16.

65. A use of claim 61, wherein the polypeptide has at least 90% sequence identity with xage-1 p16 (SEQ ID NO:4) and which, when processed and presented in the context of Major Histocompatibility Complex molecules, activates T lymphocytes against cells which express xage-1 p16.

66. A use of claim 61, wherein the cells expressing xage-1 p16 are cancer cells.

67. A use of claim 66, wherein the cancer cells are of cancers other than Ewing's sarcoma or alveolar rhabdomyosarcoma.

68. A use of claim 66, wherein the cancer cells expressing xage-1 p16 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells.

69. A use of an isolated, recombinant nucleic acid molecule comprising a nucleotide sequence encoding a polypeptide selected from the group of a polypeptide having the amino acid sequence of an xage-1 p16 protein (“p16” (SEQ ID NO:4)), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16, for the manufacture of a medicament for activating T lymphocytes against cells expressing xage-1 p16.

70. A use of claim 69, wherein said cells expressing xage-1 p16 are cancer cells.

71. A use of claim 70, wherein said cancer cells are of a cancer other than Ewing's sarcoma or alveolar rhabdomyosarcoma.

72. A use of claim 70, wherein the cells expressing xage-1 p16 are selected from the group consisting of prostate cancer cells, lung cancer cells, ovarian cancer cells, breast cancer cells, glioblastoma cells, pancreatic cancer cells, T cell lymphoma cells, melanoma cells, and histocytic lymphoma cells.

73. A use of claim 69, wherein the isolated, recombinant nucleic acid molecule encodes xage-1 p16 (SEQ ID NO:4).

74. A use of claim 69, wherein the isolated, recombinant nucleic acid molecule encodes an immunogenic fragment of xage-1 p16.

75. A use of claim 69, wherein the isolated, recombinant nucleic acid molecule encodes a polypeptide with at least 90% sequence identity to xage-1 p16 (SEQ ID NO:4) and which is specifically recognized by an antibody which specifically recognizes xage-1 p16.

76. A use of claim 69, wherein the isolated recombinant nucleic acid molecule encodes a polypeptide with at least 90% sequence identity with xage-1 p16 (SEQ ID NO:4) and which, when processed and presented in the context of Major Histocompatibility Complex molecules, activates T lymphocytes against cells which express xage-1 p16.

77. An antibody that specifically binds to an epitope of a polypeptide selected from the group consisting of an xage-1 p16 protein (SEQ ID NO:4), an immunogenic fragment thereof, a polypeptide with at least 90% sequence identity to p16 and which is specifically recognized by an antibody which specifically recognizes p16, and a polypeptide which has at least 90% sequence identity with p16 and which, when processed and presented in the context of Major Histocompatibility Complex molecules, activates T lymphocytes against cells which express p16.

78. An antibody of claim 77, wherein said protein is xage-1 p16 (SEQ ID NO:4).

79. The antibody of claim 77, further comprising a therapeutic moiety or a detectable label.

80. The antibody of claim 77, wherein the therapeutic moiety is a toxic moiety.

81. The antibody of claim 80, wherein the toxic moiety is selected from the group consisting of ricin A, abrin, ribotoxin, ribonuclease, saporin, calicheamycin, diphtheria toxin or a subunit thereof, Pseudomonas exotoxin, a cytotoxic portion thereof, a mutated Pseudomonas exotoxin, a cytotoxic portion thereof, and botulinum toxins A through F, pokeweed antiviral toxin or a cytotoxic fragment thereof, and bryodin 1 or a cytotoxic fragment thereof.

82. The antibody of claim 81, wherein the toxic moiety is a Pseudomonas exotoxin or a cytotoxic fragment thereof.

83. The antibody of claim 81, wherein the Pseudomonas exotoxin is selected from the group consisting of PE35, PE38, PE4E, and PE40.

84. The antibody of claim 79, wherein the detectable label is a radiolabel.

85. A method of inhibiting the growth of a cancer cell expressing xage-1 p16 (SEQ ID NO:4) on its exterior surface, comprising contacting the cell with an immunoconjugate comprising a therapeutic moiety and a targeting moiety, the targeting moiety comprising a polypeptide comprising an antibody which specifically binds to an epitope of xage-1 p16, wherein said binding permits the therapeutic moiety to inhibit the growth of the cell.

86. The method of claim 85, wherein the therapeutic moiety is a drug.

87. The method of claim 85, wherein the therapeutic moiety is a radioisotope.

88. The method of claim 85, wherein the therapeutic moiety is a toxin.

89. The method of claim 88, wherein the toxin is selected from the group consisting of ricin A, abrin, ribotoxin, ribonuclease, saporin, calicheamycin, diphtheria toxin or a subunit thereof, Pseudomonas exotoxin, a cytotoxic portion thereof, a mutated Pseudoinonas exotoxin, a cytotoxic portion thereof, and botulinum toxins A through F, pokeweed antiviral toxin or a cytotoxic fragment thereof, and bryodin 1 or a cytotoxic fragment thereof.

90. The method of claim 89, wherein said toxin is a modified Pseudomonas exotoxin or cytotoxic fragment thereof

91. A kit for the detection of an xage-1 p16-expressing cancer in a sample, said kit comprising a container and an antibody which specifically recognizes xage-1 p16 (SEQ ID NO:4).

92. A kit of claim 91, wherein the xage-1 p16-expressing cancer is a cancer other than Ewing's sarcoma or alveolar rhabdomyosarcoma.


SEQUENCE LISTING (11 sequences)

SEQ ID NO: 1 - DNA, 246 bp, Homo sapiens
  CDS (1)..(246): xage-1 p9
atggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaa

SEQ ID NO: 2 - protein, 81 aa, Homo sapiens
  xage-1 p9
MESPKKKNQQLKVGILHLGSRQKKIRIQLRSQCATWKVICKSCISQTPGINLDLGSGVKVKIIPKEEHCKMPEAGEEQPQV

SEQ ID NO: 3 - DNA, 441 bp, Homo sapiens
  CDS (1)..(441): xage-1 p16
atgctcctttggtgcccacctcagtgcgcatgttcactgggcgtcttcccatcggccccttcgccagtgtggggaacgcggcggagctgtgagccggcgactcgggtccctgaggtctggattctttctccgctactgagacacggcggacacacacaaacacagaaccacacagccagtcccaggagcccagtaatggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaa

SEQ ID NO: 4 - protein, 146 aa, Homo sapiens
  xage-1 p16
MLLWCPPQCACSLGVFPSAPSPVWGTRRSCEPATRVPEVWILSPLLRHGGHTQTQNHTASPRSPVMESPKKKNQQLKVGILHLGSRQKKIRIQLRSQCATWKVICKSCISQTPGINLDLGSGVKVKIIPKEEHCKMPEAGEEQPQV

SEQ ID NO: 5 - DNA, 637 bp, Homo sapiens
  complete XAGE-1 sequence with untranslated 5′ and 3′ ends
gtcgttaatggggacctgggaaggagcataggacagggcaaggcgggataaggaggggcaccacagcccttaaggcacgagggaacctcactgcgcatgctcctttggtgcccacctcagtgcgcatgttcactgggcgtcttcccatcggccccttcgccagtgtggggaacgcggcggagctgtgagccggcgactcgggtccctgaggtctggattctttctccgctactgagacacggcggacacacacaaacacagaaccacacagccagtcccaggagcccagtaatggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaaatgaagacaagctgaaacaacgcaagctggttttatattagatatttgacttaaactatctcaataaagttttgcagctttcaccaaaaaaaaaaaaaaa

SEQ ID NO: 6 - protein, 4 aa, Artificial Sequence
  Description of Artificial SequencePseudomonas exotoxin carboxy terminus addition
KDEL

SEQ ID NO: 7 - protein, 4 aa, Artificial Sequence
  Description of Artificial SequencePseudomonas exotoxin carboxy terminus addition
REDL

SEQ ID NO: 8 - DNA, 30 bp, Artificial Sequence
  Description of Artificial Sequenceprimer xa-1
cagcttgtcttcatttaaacttgtggttgc

SEQ ID NO: 9 - DNA, 23 bp, Artificial Sequence
  Description of Artificial Sequenceprimer xa-2
tcccaggagcccagtaatggaga

SEQ ID NO: 10 - DNA, 20 bp, Artificial Sequence
  Description of Artificial Sequenceprimer xa-8
acctgggaaggagcatagga

SEQ ID NO: 11 - DNA, 32 bp, Artificial Sequence
  Description of Artificial Sequenceprimer xa-10
ctttattgagatagtttaagtcaaatatctaa